(19) United States
(12) Patent Application Publication
(10) Pub. No.: US 2014/0227174 A1
(43) Pub. Date: Aug. 14, 2014

(54) SKIN PERMEATING AND CELL ENTERING (SPACE) PEPTIDES AND METHODS OF USE THEREFOR

(71) Applicants: CONVOY THERAPEUTICS, Las Vegas, NV (US); THE REGENTS OF THE UNIVERSITY OF CALIFORNIA, Oakland, CA (US)

(72) Inventors: John A. Muraski, Oro Valley, AZ (US); Samir Mitragotri, Santa Barbara, CA (US); Ming Chen, Santa Barbara, CA (US)

(73) Assignees: THE REGENTS OF THE UNIVERSITY OF CALIFORNIA, Oakland, CA (US); CONVOY THERAPEUTICS, Las Vegas, NV (US)

(21) Appl. No.: 13/764,359

(22) Filed: Feb. 11, 2013

(51) Int. Cl. A61K 47/42; A61K 31/728; A61K 38/13; A61K 47/48; A61K 31/7088

(52) U.S. Cl. CPC A61K 47/42; A61K 47/48723; A61K 31/7088; A61K 38/13; A61K 31/728; A61K 47/48246
USPC 424/1.21; 424/450; 424/499; 424/130.1; 424/178.1; 514/1.2

(57) ABSTRACT

Compositions that facilitate the delivery of an active agent or an active agent carrier wherein the compositions are capable of penetrating the stratum corneum (SC) and/or the cellular membranes of viable cells are provided. In some embodiments, the compositions include a peptide, an active agent, and a carrier that includes the active agent, wherein the peptide has an amino acid sequence set forth in any of SEQ ID NOs: 1-18; the peptide is associated with and/or conjugated to the active agent, the carrier, or both; the carrier is selected from the group consisting of a micelle, a liposome, an ethosome, and combinations thereof; and the composition is capable of penetrating a stratum corneum (SC) layer when contacted therewith or penetrating a cell when contacted therewith, and optionally wherein the composition further includes one or more free peptides having an amino acid sequence set forth in any of SEQ ID NOs: 1-18. Also provided are methods for delivering active agents to subjects, methods for treating subjects having dermatological diseases, and methods for attenuating expression of mRNAs of subjects in need thereof and/or for treating diseases and/or disorders thereby.

### TECHNICAL FIELD

The presently disclosed subject matter relates to peptides, optionally peptides conjugated to one or more active agents and/or active agent carriers comprising the active agent(s). Also provided are compositions comprising the presently disclosed peptides and/or conjugates, wherein the compositions are capable of penetrating a stratum corneum (SC) layer when contacted therewith or penetrating a cell when contacted therewith, as well as methods for employing the claimed peptides, conjugates, and/or compositions to deliver active agents to subjects.

### BACKGROUND

Skin, the largest organ of the human body, is a host to numerous dermatological diseases which collectively represent a large category of human health conditions. Accordingly, successful delivery of therapeutics, e.g., macromolecules such as siRNA, into skin has become a topic of active research and development. The goal of topical siRNA delivery, however, is extremely challenging and with some exceptions, has been very difficult to accomplish. The primary challenge is poor skin penetration of macromolecules. Among various physico-chemical methods proposed to enhance penetration of macromolecules, peptide carriers have emerged as potential candidates owing to their simplicity of use, diversity and potential ability to target cellular sub-types within the skin. Several peptides including TAT, polyarginine, meganin, and penetratin, which were initially identified for delivering drugs into the cytoplasm of cells, have been tested for penetration across the stratum corneum (SC) and a few have shown some efficacy in delivering small molecules into the epidermis. In contrast, only one peptide, TD-1, has been specifically shown to penetrate the SC and possess the ability to enhance systemic uptake of topically applied drugs. Although several peptides are known to penetrate cellular membranes and a few to penetrate the SC, peptides that simultaneously enhance the penetration of macromolecules and other actives across the SC and/or across the cellular membranes of viable epidermal and dermal cells are needed.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter provides compositions comprising a peptide, an active agent, and a carrier comprising the active agent. In some embodiments, the peptide comprises an amino acid sequence set forth in any of SEQ ID NOs: 1-18; the peptide is associated with and/or conjugated to the active agent, the carrier, or both; the carrier is selected from the group consisting of a micelle, a liposome, an ethosome, and combinations thereof; and/or the composition is capable of penetrating a stratum corneum (SC) layer when contacted therewith or penetrating a cell when contacted therewith, and optionally wherein the composition further comprises one or more free peptides comprising an amino acid sequence set forth in any of SEQ ID NOs: 1-18. In some embodiments, the composition is capable of penetrating the SC layer and penetrating the cell. In some embodiments, the peptide is a cyclic peptide comprising an amino acid sequence as set forth in any of SEQ ID NOs: 7-18 and a Cys-Cys disulfide bond.

In some embodiments, the composition is capable of penetrating the cellular membrane of viable non-human animal cells; viable human cells; viable epidermal or dermal cells; and/or viable immunological cells.

In some embodiments, the active agent comprises a macromolecule, optionally a protein, a nucleic acid, a pharmaceutical compound, a detectable moiety, a small molecule, and/or a nanoparticle. In some embodiments, the protein comprises an antibody or a fragment thereof comprising at least one paratope. In some embodiments, the macromolecule comprises a nucleic acid, optionally DNA or RNA, and further optionally wherein the nucleic acid is an interfering RNA, an shRNA, an miRNA, or an siRNA. In some embodiments, the siRNA is designed to interfere with expression of a gene product selected from the group consisting of an IL-10 gene product, an IL-4 gene product, an CD86 gene product, a KRT6a gene product, a TNFR1 gene product, and a TACE gene product. In some embodiments, the siRNA is a mutation-specific siRNA.

In some embodiments, the pharmaceutical compound is cyclosporin A (CsA) or hyaluronic acid (HA). In some embodiments, the pharmaceutical compound is CsA, the CsA is encapsulated by the carrier, and the peptide is conjugated to the carrier. In some embodiments, the carrier is an ethosome and the composition further comprises one or more free peptides comprising an amino acid sequence set forth in any of SEQ ID NOs: 1-18.

In some embodiments, the active agent comprises a detectable agent, optionally a fluorescent label or a radioactive label.

In some embodiments, the presently disclosed subject matter also provides compositions comprising a peptide, an active agent, and a carrier comprising the active agent, wherein the peptide comprises an amino acid sequence set forth in any of SEQ ID NOs: 1-18; the peptide is associated with an active agent and/or a carrier comprising the active agent, wherein the association results from hydrophobic, electrostatic or van der Walls interactions; the carrier is selected from the group consisting of a micelle, a liposome, an ethosome, and combinations thereof; and the composition is capable of penetrating a stratum corneum (SC) layer when contacted therewith or penetrating a cell when contacted therewith, and further wherein the composition optionally comprises one or more free peptides comprising an amino acid sequence set forth in any of SEQ ID NOs: 1-18. In some embodiments, the peptide is a cyclic peptide comprising (i) an amino acid sequence as set forth in any of SEQ ID NOs: 7-18, and (ii) a Cys-Cys disulfide bond.

The presently disclosed subject matter also provides in some embodiments methods for delivering an active agent to a subject. In some embodiments, the methods comprise administering to the subject a composition comprising a peptide comprising an amino acid sequence set forth in any of SEQ ID NOs: 1-18, wherein the peptide is conjugated to an active agent or an active agent carrier comprising the active agent and/or is associated with an active agent and/or a carrier comprising the active agent, wherein the association results from hydrophobic, electrostatic or van der Walls interactions; the carrier is selected from the group consisting of a micelle, a liposome, an ethosome, and combinations thereof; and the composition is capable of penetrating the stratum corneum (SC) of the subject or penetrating a cell of the subject, and optionally wherein the composition further comprises one or more free peptides comprising an amino acid sequence set forth in any of SEQ ID NOs: 1-18.

In some embodiments, the composition is formulated for topical administration.

In some embodiments, the peptide is a cyclic peptide comprising an amino acid sequence as set forth in any of SEQ ID NOs: 7-18 and a Cys-Cys disulfide bond.

In some embodiments, the composition is capable of penetrating the cellular membrane of viable non-human animal cells, viable human cells, viable epidermal cells, viable dermal cells, and/or viable immunological cells.

In some embodiments, the active agent comprises a macromolecule, optionally a protein, a nucleic acid, a pharmaceutical compound, a detectable moiety, a small molecule, and/or a nanoparticle. In some embodiments, the protein comprises an antibody or a fragment thereof comprising at least one paratope. In some embodiments, the macromolecule comprises a nucleic acid, optionally a DNA molecule. In some embodiments, the nucleic acid is RNA, optionally an interfering RNA, further optionally an shRNA, an miRNA, or an siRNA. In some embodiments, the siRNA is designed to interfere with expression of a gene product selected from the group consisting of an IL-10 gene product, an IL-14 gene product, an CD86 gene product, a KRT6a gene product, a TNFR1 gene product, and a TACE gene product. In some embodiments, the siRNA is a mutation-specific siRNA.

In some embodiments, the pharmaceutical compound is cyclosporin A (CsA) or hyaluronic acid (HA). In some embodiments, the pharmaceutical compound is CsA, the CsA is encapsulated by the carrier, and the peptide is conjugated to the carrier. In some embodiments, the carrier is an ethosome and the composition further comprises one or more free peptides comprising an amino acid sequence set forth in any of SEQ ID NOs: 1-18.

The presently disclosed subject matter also provides in some embodiments methods for treating a subject having a dermatological disease. In some embodiments, the methods comprise administering to the subject a composition comprising a peptide comprising an amino acid sequence set forth in any of SEQ ID NOs: 1-18, wherein the peptide is conjugated to an active agent or an active agent carrier comprising the active agent and/or is associated with an active agent and/or a carrier comprising the active agent, wherein the association results from hydrophobic, electrostatic or van der Walls interactions; the carrier is selected from the group consisting of a micelle, a liposome, an ethosome, and combinations thereof; and the composition is capable of penetrating the stratum corneum (SC) of the subject or penetrating a cell of the subject, and optionally wherein the composition further comprises one or more free peptides comprising an amino acid sequence set forth in any of SEQ ID NOs: 1-18.

In some embodiments, the composition is formulated for topical administration.

In some embodiments, the peptide is a cyclic peptide comprising (i) an amino acid sequence as set forth in any of SEQ ID NOs: 7-18; and (ii) a Cys-Cys disulfide bond.

In some embodiments, the composition is capable of penetrating the cellular membrane of viable non-human animal cells, viable human cells, viable epidermal cells, viable dermal cells, and/or viable immunological cells.

In some embodiments, the active agent comprises a macromolecule, optionally a protein, a nucleic acid, a pharmaceutical compound, a detectable moiety, a small molecule, and/or a nanoparticle. In some embodiments, the protein comprises an antibody or a fragment thereof comprising at least one paratope. In some embodiments, the macromolecule comprises a nucleic acid, optionally a DNA molecule. In some embodiments, the nucleic acid is RNA, optionally an interfering RNA, further optionally an shRNA, an miRNA, or an siRNA. In some embodiments, the siRNA is designed to interfere with expression of a gene product selected from the group consisting of an IL-10 gene product, an IL-14 gene product, an CD86 gene product, a KRT6a gene product, a TNFR1 gene product, and a TACE gene product. In some embodiments, the siRNA is a mutation-specific siRNA.

In some embodiments, the pharmaceutical compound is cyclosporin A (CsA) or hyaluronic acid (HA). In some embodiments, the pharmaceutical compound is CsA, the CsA is encapsulated by the carrier, and the peptide is conjugated to the carrier. In some embodiments, the carrier is an ethosome and the composition further comprises one or more free peptides comprising an amino acid sequence set forth in any of SEQ ID NOs: 1-18.

The presently disclosed subject matter also provides methods for treating a subject having, suspected of having, and/or susceptible to a disorder resulting at least in part from expression of an mRNA. In some embodiments, the methods comprise administering to the subject a composition comprising a peptide comprising an amino acid sequence set forth in any of SEQ ID NOs: 1-18, wherein the peptide is conjugated to an interfering RNA which targets the mRNA or an active agent carrier comprising an interfering RNA which targets the mRNA and/or is associated with an interfering RNA which targets the mRNA and/or a carrier comprising an interfering RNA which targets the mRNA, wherein the association results from hydrophobic, electrostatic or van der Walls interactions; the carrier is selected from the group consisting of a micelle, a liposome, an ethosome, and combinations thereof; and the composition is capable of penetrating the stratum corneum (SC) of the subject or penetrating a cell of the subject, and optionally wherein the composition further comprises one or more free peptides comprising an amino acid sequence set forth in any of SEQ ID NOs: 1-18.

In some embodiments, the composition is formulated for topical administration. In some embodiments, the composition is capable of penetrating the cellular membrane of viable non-human animal cells, viable human cells, viable epidermal cells, viable dermal cells, and/or viable immunological cells.

In some embodiments, the peptide is a cyclic peptide comprising (i) an amino acid sequence as set forth in any of SEQ ID NOs: 7-18; and (ii) a Cys-Cys disulfide bond.

In some embodiments, the active agent comprises a macromolecule, optionally a protein, a nucleic acid, a pharmaceutical compound, a detectable moiety, a small molecule, and/or a nanoparticle. In some embodiments, the protein comprises an antibody or a fragment thereof comprising at least one paratope. In some embodiments, the macromolecule comprises a nucleic acid, optionally a DNA molecule. In some embodiments, the nucleic acid is RNA, optionally an interfering RNA, further optionally an shRNA, an miRNA, or an siRNA. In some embodiments, the siRNA is designed to interfere with expression of a gene product selected from the group consisting of an IL-10 gene product, an CD86 gene product, a KRT6a gene product, a TNFR1 gene product, and a TACE gene product. In some embodiments, the siRNA is a mutation-specific siRNA.

In some embodiments, the pharmaceutical compound is cyclosporin A (CsA) or hyaluronic acid (HA). In some embodiments, the pharmaceutical compound is CsA, the CsA is encapsulated by the carrier, and the peptide is conjugated to the carrier. In some embodiments, the carrier is an ethosome and the composition further comprises one or more free peptides comprising an amino acid sequence set forth in any of SEQ ID NOs: 1-18.

The presently disclosed subject matter also provides in some embodiments methods for attenuating expression of an mRNA of a subject in need thereof. In some embodiments, the methods comprise administering to the subject a composition comprising a peptide comprising an amino acid sequence set forth in any of SEQ ID NOs: 1-18, wherein the peptide is conjugated to an interfering RNA which targets the mRNA or an active agent carrier comprising an siRNA which targets the mRNA and/or is associated with an siRNA which targets the mRNA and/or a carrier comprising an siRNA which targets the mRNA, wherein the association results from hydrophobic, electrostatic or van der Walls interactions; the carrier is selected from the group consisting of a micelle, a liposome, an ethosome, and combinations thereof; and the composition is capable of penetrating the stratum corneum (SC) of the subject or penetrating a cell of the subject, and optionally wherein the composition further comprises one or more free peptides comprising an amino acid sequence set forth in any of SEQ ID NOs: 1-18. In some embodiments, the peptide is a cyclic peptide comprising an amino acid sequence as set forth in any of SEQ ID NOs: 7-18 and a Cys-Cys disulfide bond.

In some embodiments, the composition is formulated for topical administration. In some embodiments, the composition is capable of penetrating the cellular membrane of viable non-human animal cells, viable human cells, viable epidermal cells, viable dermal cells, and/or viable immunological cells.

In some embodiments, the active agent comprises a macromolecule, optionally a protein, a nucleic acid, a pharmaceutical compound, a detectable moiety, a small molecule, and/or a nanoparticle. In some embodiments, the protein comprises an antibody or a fragment thereof comprising at least one paratope. In some embodiments, the macromolecule comprises a nucleic acid, optionally a DNA molecule. In some embodiments, the nucleic acid is RNA, optionally an interfering RNA, further optionally an shRNA, an miRNA, or an siRNA. In some embodiments, the siRNA is designed to interfere with expression of a gene product selected from the group consisting of an IL-10 gene product, an CD86 gene product, a KRT6a gene product, a TNFR1 gene product, and a TACE gene product. In some embodiments, the siRNA is a mutation-specific siRNA.

In some embodiments, the pharmaceutical compound is cyclosporin A (CsA) or hyaluronic acid (HA). In some embodiments, the pharmaceutical compound is CsA, the CsA is encapsulated by the carrier, and the peptide is conjugated to the carrier. In some embodiments, the carrier is an ethosome and the composition further comprises one or more free peptides comprising an amino acid sequence set forth in any of SEQ ID NOs: 1-18.

The presently disclosed subject matter also provides in some embodiments compositions comprising a peptide an active agent, and a carrier comprising the active agent. In some embodiments, the peptide consists essentially of an amino acid sequence set forth in any of SEQ ID NOs: 1-18; the peptide is conjugated to the active agent, the carrier, or both; the carrier is selected from the group consisting of a micelle, a liposome, an ethosome, and combinations thereof; and/or the composition is capable of penetrating a stratum corneum (SC) layer when contacted therewith or penetrating a cell when contacted therewith. In some embodiments, the composition optionally comprises one or more free peptides comprising an amino acid sequence set forth in any of SEQ ID NOs: 1-18.

The presently disclosed subject matter also provides in some embodiments the presently disclosed compositions formulated for use in a cosmetic preparation. In some embodiments, the formulated composition has a pH of from about 2 to about 10, optionally of from about 4 to about 8.

Thus, it is an object of the presently disclosed subject matter to provide compositions and methods for delivering active agents to subjects.

An object of the presently disclosed subject matter having been stated hereinabove, and which is achieved in whole or in part by the presently disclosed subject matter, other objects will become evident as the description proceeds when taken in connection with the accompanying drawings as best described herein below.

### BRIEF DESCRIPTION OF THE DRAWINGS

FIG. 1 is an exemplary scheme for conjugating a SPACE Peptide of the presently disclosed subject matter to Cyclosporin A (CsA) employing a CsA epoxide intermediate.

FIGS. 2A-2F are infrared (IR) spectra (FIGS. 2A-2E) and mass spectrometry (FIG. 2F) traces of reactants, intermediates, and products from an exemplary synthesis using the scheme of FIG. 1. FIG. 2A is an IR spectrum trace of the SPACE Peptide powder showing a characteristic absorption band at about 700-800 cm⁻¹ (circled). FIG. 2B is an IR spectrum trace of the CsA powder showing a characteristic absorption band at about 2900 cm⁻¹ (circled). FIG. 2C is an IR spectrum trace of the conjugation powder showing characteristic SPACE Peptide absorption band at about 700-800 cm⁻¹ (circled) and the characteristic CsA absorption band at 2900 cm⁻¹ (circled). FIG. 2D is a comparison of IR spectrum traces comparing the SPACE Peptide and final conjugation products showing that the conjugation product has the same characteristic absorption band as the SPACE Peptide does (circled area on right side of FIG. 2D). The circled area on the left side of FIG. 2D is the characteristic CyA absorption band present in the CsA-SPACE trace that is absent in the SPACE Peptide trace. FIG. 2E is a comparison of IR spectrum traces comparing the CsA and final conjugation products showing that the conjugation product also has the same characteristic absorption band as does CsA (circled area on the left side of FIG. 2E). The circled area on the right side of FIG. 2E shows that CsA does not have a band characteristic of SPACE peptide. FIG. 2F is a mass spectrometry trace of reactants (SPACE Peptide (SPACE) and an epoxide of cyclosporin A (CsA-Epoxide)) and a product (SPACE Peptide-conjugated cyclosporin A (CsA-SP)) of the presently disclosed subject matter.

FIGS. 3A and 3B are schematic diagrams of in vitro skin penetration tests that can be employed for testing the abilities of the compositions of the presently disclosed subject matter to deliver active agents to various layers of the skin.

FIG. 4 is a series of bar graphs showing the results of employing a SPACE Peptide of the presently disclosed subject matter to deliver a fluorescent label (FITC) to various layers of the skin.

FIG. 5 is a schematic representation of the measurement of partitioning of SPACE peptide. FITC-SPACE peptide was incubated epidermis-SC and dermis for 48 hours at 37° C. and the amount of SPACE peptide partitioned into the tissue was measured.

FIG. 6 is a schematic diagram of an exemplary generalized method for preparing SPACE Peptide/lipid conjugates.

FIGS. 7A and 7B are a schematic diagram of an exemplary generalized method for preparing SPACE Peptide/lipid conjugates and SPACE Peptide-conjugated ethosomes. FIG. 7A presents steps where a SPACE Peptide in PBS is mixed with POPE-NHS in ethanol. To this mixture is added lipids in ethanol and FITC, which produces lipids conjugated with SPACE Peptide in a PBS/Ethanol mixture. FIG. 7B presents step wherein the ethanol is removed from the mixture of lipids conjugated with the SPACE Peptide either completely or to a degree necessary to adjust the ethanol:PBS ratio to a desired point prior to repeated extrusion through a 100 nm membrane, which results in the recovery of either liposomes or ethosomes conjugated with SPACE Peptide. It is noted that in FIGS. 7A and 7B, the specific concentrations listed are exemplary only and are not intended to limit the generalized methods depicted.

FIGS. 8A-8C are a series of bar graphs depicting the results of delivery of different SPACE-associated cargos to the skin and different compartments thereof. FIG. 8A is a bar graph depicting delivery of FITC by a composition containing a FITC-conjugated SPACE Peptide (bar A); a composition containing a FITC-conjugated SPACE Peptide in conjunction with free SPACE Peptide (bar B); a composition containing a SPACE Peptide-conjugated liposome encapsulating FITC (bar C); a composition containing a SPACE Peptide-conjugated ethosome encapsulating FITC (bar D); and a composition containing a SPACE Peptide-conjugated ethosome encapsulating FITC in conjunction with free SPACE Peptide (bar E). FIG. 8B is a bar graph depicting delivery of FITC by Compositions A-E of FIG. 8A to various layers of the skin. In each of the five sets of bars, the delivery to SC-1, SC-2, SC-3, epidermis, dermis, and the receiver, left to right respectively, is depicted. FIG. 8C is a bar graph showing delivery of a fluorescent label to EPIDERM™ (blue; left column of each pair) and a pig skin model (pink; right column of each pair) using different formulations the contain SPACE Peptides. As shown in the Figure, a FITC-labeled SPACE Peptide penetrated into EPIDERM™, encapsulation of the FITC-labeled SPACE Peptide into a SPACE Peptide-conjugated ethosome further enhanced penetration in to EPIDERM™, and the addition of a free SPACE Peptide into the formulation further enhanced skin penetration both in EPIDERM™ and in the pig skin model.

FIG. 9 is a depiction of an exemplary SPACE Peptide-conjugated ethosome that, in some embodiments, can be employed to deliver cyclosporin A to the skin. The general characteristics for the SPACE Peptide-conjugated ethosome listed in the Figure are intended to be exemplary only.

FIG. 10 is a bar graph depicting delivery of cyclosporin A (CsA) encapsulated in an exemplary SPACE Peptide-conjugated ethosome composition (Etsm/CsA) into various layers of the skin. In this example, the concentration of CsA in the skin was 1284-fold higher than in the receiver fluid.

FIGS. 11A-11D are a series of bar graphs depicting delivery of hyaluronic acid (HA) using the SPACE-Peptide conjugates of the presently disclosed subject matter. FIG. 11A is a bar graph depicting delivery of HA encapsulated in a SPACE Peptide-conjugated ethosome (Etsm/HA) to various skin layers using HA encapsulated in a SPACE Peptide-conjugated ethosome (Etsm/HA). FIG. 11B is a bar graph comparing delivery of HA encapsulated in a SPACE Peptide-conjugated ethosome (Etsm/HA) to various skin layers in the presence or absence of Free SPACE Peptides. The numbers in parentheses in the x-axis labels show the concentration of free SPACE Peptide included in the formulations. FIG. 11C is a bar graph comparing delivery of HA encapsulated in a SPACE Peptide-conjugated ethosome (Etsm/HA) to various skin layers in the presence or absence of Free SPACE Peptides. The numbers in parentheses in the x-axis labels show the concentration of SPACE-lipid employed in the formulations. FIG. 11D is a bar graph comparing delivery of HA encapsulated in a SPACE Peptide-conjugated ethosome (Etsm/HA) to various skin layers in the presence or absence of Free SPACE Peptides at pH 4 or at pH 8.

FIGS. 12A and 12B are bar graphs depicting delivery of an exemplary siRNA encapsulated in a SPACE Peptide-conjugated ethosome. In each of FIGS. 12A and 12B, the y-axis presents a percentage of the 100 μl applied dose that was found in the indicated layers 24 hours after application to a 2 cm² sample after 24 hours.

### BRIEF DESCRIPTION OF THE SEQUENCE LISTING

SEQ ID NOs: 1-18 are the amino acid sequences of eighteen (18) exemplary SPACE Peptides. In some embodiments of SPACE Peptides having the amino acid sequences of SEQ ID NOs: 7-18, the SPACE Peptides are cyclic peptides that include an intrapeptide Cys-Cys disulfide bond.

SEQ ID NO. 19 is a nucleotide sequence of an siRNA that is targeted to glyceraldehyde 3-phosphate dehydrogenase (GAPDH). In some embodiments, SEQ ID NO. 19 is modified at the 5′-terminus, the 3′-terminus, or both.

### DETAILED DESCRIPTION

### I. Definitions

Before the presently disclosed subject matter is further described, it is to be understood that the presently disclosed subject matter is not limited to particular embodiments described, as such can, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the presently disclosed subject matter. The upper and lower limits of these smaller ranges can independently be included in the smaller ranges, and are also encompassed within the presently disclosed subject matter, subject to any specifically excluded limit in the stated range. Where the stated range includes one or both of the limits, ranges excluding either or both of those included limits are also included in the presently disclosed subject matter.

Unless defined otherwise, all technical and scientific terms used herein have the same meaning as commonly understood by one of ordinary skill in the art to which the presently disclosed subject matter belongs. Although any methods and materials similar or equivalent to those described herein can also be used in the practice or testing of the presently disclosed subject matter, exemplary methods and materials are now described. All publications and applications mentioned herein are incorporated by reference to disclose and describe the methods and/or materials in connection with which the publications are cited. To the extent any of the applications or publications incorporated by reference herein conflict with the instant disclosure, the instant disclosure controls.

It must be noted that as used herein and in the appended claims, the singular forms “a”, “and”, and “the” include plural referents unless the context clearly dictates otherwise. Thus, for example, reference to “a peptide” includes a plurality of such peptides and reference to the “agent” includes reference to one or more agents and equivalents thereof known to those skilled in the art, and so forth.

The publications discussed herein are provided solely for their disclosure prior to the filing date of the present application. Nothing herein is to be construed as an admission that the presently disclosed subject matter is not entitled to antedate such publication by virtue of prior conception and/or reduction to practice. Further, the dates of publication provided can be different from the actual publication dates, which might need to be independently confirmed.

It will be appreciated that throughout this present disclosure reference is made to amino acids according to the single letter or three letter codes. For convenience, the single and three letter codes for each amino acid, as well as functionally equivalent codons therefor, are provided below in Table 1:

TABLE 1
Amino Acid Abbreviations, Codes, and Functionally Equivalent Codons

| Amino Acid | 3-Letter | 1-Letter | Codons |
|---|---|---|---|
| Alanine | Ala | A | GCA GCC GCG GCU |
| Arginine | Arg | R | AGA AGG CGA CGC CGG CGU |
| Asparagine | Asn | N | AAC AAU |
| Aspartic Acid | Asp | D | GAC GAU |
| Cysteine | Cys | C | UGC UGU |
| Glutamic acid | Glu | E | GAA GAG |
| Glutamine | Gln | Q | CAA CAG |
| Glycine | Gly | G | GGA GGC GGG GGU |
| Histidine | His | H | CAC CAU |
| Isoleucine | Ile | I | AUA AUC AUU |
| Leucine | Leu | L | UUA UUG CUA CUC CUG CUU |
| Lysine | Lys | K | AAA AAG |
| Methionine | Met | M | AUG |
| Phenylalanine | Phe | F | UUC UUU |
| Proline | Pro | P | CCA CCC CCG CCU |
| Serine | Ser | S | ACG AGU UCA UCC UCG UCU |
| Threonine | Thr | T | ACA ACC ACG ACU |
| Tryptophan | Trp | W | UGG |
| Tyrosine | Tyr | Y | UAC UAU |
| Valine | Val | V | GUA GUC GUG GUU |

As used herein, the term “active agent” refers to an agent, e.g., a protein, peptide, nucleic acid (including, e.g., nucleotides, nucleosides and analogues thereof) or small molecule drug, that provides a desired pharmacological effect upon administration to a subject, e.g., a human or a non-human animal, either alone or in combination with other active or inert components. Included in the above definition are precursors, derivatives, analogues and prodrugs of active agents. The term “active agent” can also be used herein to refer generally to any agent, e.g., a protein, peptide, nucleic acid (including, e.g., nucleotides, nucleosides and analogues thereof) or small molecule drug, conjugated or associated with a penetrating peptide as described herein or attached to or encompassed by an active agent carrier as described herein.

The term “conjugated” as used in the context of the penetrating peptide compositions described herein refers to a covalent or ionic interaction between two entities, e.g., molecules, compounds or combinations thereof.

The term “associated” as used in the context of the penetrating peptide compositions described herein refers to a non-covalent interaction between two entities, e.g., molecules, compounds or combinations thereof mediated by one or more of hydrophobic, electrostatic, and van der Walls interactions.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and native leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

The terms “antibody” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins including an antigen-binding portion of an antibody and a non-antibody protein. The antibodies can be detectably labeled, e.g., with a radioisotope, an enzyme which generates a detectable product, a fluorescent protein, and the like. The antibodies can be further conjugated to other moieties, such as members of specific binding pairs, e.g., biotin (member of biotin-avidin specific binding pair), and the like. Also encompassed by the terms are Fab′, Fv, F(ab′)₂, and other antibody fragments that retain specific binding to antigen.

Antibodies can exist in a variety of other forms including, for example, Fv, Fab, and (Fab′)₂, as well as bi-functional (i.e., bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). See generally, Hood et al., Immunology, Benjamin, N.Y., 2nd ed. (1984), and Hunkapiller and Hood, Nature, 323, 15-16 (1986).

The terms “nucleic acid”, “nucleic acid molecule” and “polynucleotide” are used interchangeably and refer to a polymeric form of nucleotides of any length, either deoxyribonucleotides or ribonucleotides, or analogs thereof. The terms encompass, e.g., DNA, RNA and modified forms thereof. Polynucleotides can have any three-dimensional structure, and can perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule can be linear or circular.

“RNA interference” (RNAi) is a process by which double-stranded RNA (dsRNA) is used to silence gene expression. Without intending to be bound by any particular theory, RNAi begins with the cleavage of longer dsRNAs into small interfering RNAs (siRNAs) by dicer, an RNase III-like enzyme. siRNAs are dsRNAs that are generally about 19 to 28 nucleotides, or 20 to 25 nucleotides, or 21 to 23 nucleotides in length and often contain 2-3 nucleotide 3′ overhangs, and 5′ phosphate and 3′ hydroxyl termini. One strand of the siRNA is incorporated into a ribonucleoprotein complex known as the RNA-induced silencing complex (RISC). RISC uses this siRNA strand to identify mRNA molecules that are at least partially complementary to the incorporated siRNA strand, and then cleaves these target mRNAs or inhibits their translation. The siRNA strand that is incorporated into RISC is known as the guide strand or the antisense strand. The other siRNA strand, known as the passenger strand or the sense strand, is eliminated from the siRNA and is at least partially homologous to the target mRNA. Those of skill in the art will recognize that, in principle, either strand of an siRNA can be incorporated into RISC and function as a guide strand. However, siRNA can be designed (e.g., via decreased siRNA duplex stability at the 5′ end of the antisense strand) to favor incorporation of the antisense strand into RISC.

RISC-mediated cleavage of mRNAs having a sequence at least partially complementary to the guide strand leads to a decrease in the steady state level of that mRNA and of the corresponding protein encoded by the mRNA. Alternatively, RISC can also decrease expression of the corresponding protein via translational repression without cleavage of the target mRNA. Other RNA molecules can interact with RISC and silence gene expression. Examples of other RNA molecules that can interact with RISC include short hairpin RNAs (shRNAs), single-stranded siRNAs, microRNAs (miRNAs), and dicer-substrate 27-mer duplexes, RNA molecules containing one or more chemically modified nucleotides, one or more deoxyribonucleotides, and/or one or more non-phosphodiester linkages. The term “siRNA” as used herein refers to a double-stranded interfering RNA unless otherwise noted. For purposes of the present discussion, all RNA molecules that can interact with RISC and participate in RISC-mediated changes in gene expression will be referred to as “interfering RNAs.” siRNAs, shRNAs, miRNAs, and dicer-substrate 27-mer duplexes are, therefore, subsets of “interfering RNAs.

A “substitution” results from the replacement of one or more amino acids or nucleotides by different amino acids or nucleotides, respectively as compared to an amino acid sequence or nucleotide sequence of a polypeptide. If a substitution is conservative, the amino acid that is substituted into a polypeptide has similar structural or chemical properties (which can include, but are not limited to charge, polarity, hydrophobicity, and the like) to the amino acid that it is substituting. Conservative substitutions of naturally occurring amino acids usually result in a substitution of a first amino acid with second amino acid from the same group as the first amino acid, where exemplary amino acid groups are as follows: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, tyrosine, asparagine, and glutamine; and (4) neutral non-polar amino acids such as alanine, leucine, isoleucine, valine, proline, phenylalanine, tryptophan, and methionine. In some embodiments, polypeptide variants can have “non-conservative” changes, where the substituted amino acid differs in structural and/or chemical properties.

A “deletion” is defined as a change in either amino acid or nucleotide sequence in which one or more amino acid or nucleotide residues, respectively, are absent as compared to an amino acid sequence or nucleotide sequence of a naturally occurring polypeptide. In the context of a polypeptide or polynucleotide sequence, a deletion can involve deletion of 2, 5, 10, up to 20, up to 30, or up to 50 or more amino acids, taking into account the length of the polypeptide or polynucleotide sequence being modified, if desired.

An “insertion” or “addition” is that change in an amino acid or nucleotide sequence which has resulted in the addition of one or more amino acid or nucleotide residues, respectively, as compared to an amino acid sequence or nucleotide sequence of a naturally occurring polypeptide. “Insertion” generally refers to addition to one or more amino acid residues within an amino acid sequence of a polypeptide, while “addition” can be an insertion or refer to amino acid residues added at the N- or C-termini. In the context of a polypeptide or polynucleotide sequence, an insertion or addition can be of up to 10, up to 20, up to 30, or up to 50 or more amino acids.

“Non-native”, “non-endogenous”, and “heterologous”, in the context of a polypeptide, are used interchangeably herein to refer to a polypeptide having an amino acid sequence or, in the context of an expression system or a viral particle, present in an environment different to that found in nature.

“Exogenous” in the context of a nucleic acid or polypeptide is used to refer to a nucleic acid or polypeptide that has been introduced into a host cell. “Exogenous” nucleic acids and polypeptides can be native or non-native to the host cell, where an exogenous, native nucleic acid or polypeptide provides for elevated levels of the encoded gene product or polypeptide in the recombinant host cell relative to that found in the host cell prior to introduction of the exogenous molecule.

As used herein, the terms “determining”, “measuring”, “assessing”, and “assaying” are used interchangeably and include both quantitative and qualitative determinations.

As used herein the term “isolated”, when used in the context of an isolated compound, refers to a compound of interest that is in an environment different from that in which the compound naturally occurs. “Isolated” is meant to include compounds that are within samples that are substantially enriched for the compound of interest and/or in which the compound of interest is partially or substantially purified.

As used herein, the term “substantially pure” refers to a compound that is removed from its natural environment and is at least 60% free, 75% free, or 90% free from other components with which it is naturally associated.

A “coding sequence” or a sequence that “encodes” a selected polypeptide, is a nucleic acid molecule which is transcribed (in the case of DNA) and translated (in the case of mRNA) into a polypeptide, for example, in vivo when placed under the control of appropriate regulatory sequences (or “control elements”). The boundaries of the coding sequence are typically determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and synthetic DNA sequences. A transcription termination sequence can be located 3′ to the coding sequence. Other “control elements” can also be associated with a coding sequence. A DNA sequence encoding a polypeptide can be optimized for expression in a selected cell by using the codons preferred by the selected cell to represent the DNA copy of the desired polypeptide coding sequence.

“Encoded by” refers to a nucleic acid sequence which codes for a gene product, such as a polypeptide. Where the gene product is a polypeptide, the polypeptide sequence or a portion thereof contains an amino acid sequence of at least 3 to 5 amino acids, 8 to 10 amino acids, or at least 15 to 20 amino acids from a polypeptide encoded by the nucleic acid sequence.

“Operably linked” refers to an arrangement of elements wherein the components so described are configured so as to perform their usual function. In the case of a promoter, a promoter that is operably linked to a coding sequence will have an effect on the expression of a coding sequence. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

By “nucleic acid construct” it is meant a nucleic acid sequence that has been constructed to comprise one or more functional units not found together in nature. Examples include circular, linear, double-stranded, extrachromosomal DNA molecules (plasmids), cosmids (plasmids containing COS sequences from lambda phage), viral genomes including non-native nucleic acid sequences, and the like.

A “vector” is capable of transferring gene sequences to target cells. Typically, “vector construct”, “expression vector”, and “gene transfer vector”, mean any nucleic acid construct capable of directing the expression of a gene of interest and which can transfer gene sequences to target cells, which can be accomplished by genomic integration of all or a portion of the vector, or transient or inheritable maintenance of the vector as an extrachromosomal element. Thus, the term includes cloning, and expression vehicles, as well as integrating vectors.

An “expression cassette” includes any nucleic acid construct capable of directing the expression of a gene/coding sequence of interest, which is operably linked to a promoter of the expression cassette. Such cassettes can be constructed into a “vector”, “vector construct”, “expression vector”, or “gene transfer vector”, in order to transfer the expression cassette into target cells. Thus, the term includes cloning and expression vehicles, as well as viral vectors.

Techniques for determining nucleic acid and amino acid “sequence identity” are known in the art. Typically, such techniques include determining the nucleotide sequence of the mRNA for a gene and/or determining the amino acid sequence encoded thereby, and comparing these sequences to a second nucleotide or amino acid sequence. In general, “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics, 2: 482-489 (1981). This algorithm can be applied to amino acid sequences by using the scoring matrix developed by Dayhoff, Atlas of Protein Sequences and Structure, M. O. Dayhoff ed., 5 suppl. 3: 353-358, National Biomedical Research Foundation, Washington, D.C., USA, and normalized by Gribskov, Nucl. Acids Res. 14(6): 6745-6763 (1986).

An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995; available from Genetics Computer Group, Madison, Wis. and/or Accelrys, Inc., San Diego, Calif.). Another method of establishing percent identity in the context of the presently disclosed subject matter is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by ═HIGH SCORE; Databases=non-redundant, GENBANK®+EMBL+DDBJ+PDB+GENBANK® CDS translations+Swiss protein+Spupdate+PIR. Details of these programs can be found at the internet address located by placing http:// in front of blast.ncbi.nlm.nih.gov/Blast.cgi.

Alternatively, in the context of polynucleotides, homology can be determined by hybridization of polynucleotides under conditions that form stable duplexes between homologous regions, followed by digestion with single-stranded-specific nuclease(s), and size determination of the digested fragments.

Two DNA, or two polypeptide sequences are “substantially homologous” to each other when the sequences exhibit at least about 80%-85%, at least about 85%-90%, at least about 90%-95%, or at least about 95%-98% sequence identity over a defined length of the molecules, as determined using the methods above. As used herein, substantially homologous also refers to sequences showing complete identity to the specified DNA or polypeptide sequence. DNA sequences that are substantially homologous can be identified in a Southern hybridization experiment under, for example, stringent conditions, as defined for that particular system. Defining appropriate hybridization conditions is within the skill of the art. See e.g., Sambrook and Russell, Molecular Cloning: A Laboratory Manual, Third Edition, (2001) Cold Spring Harbor Laboratory Press, Cold Spring Harbor, N.Y.

A first polynucleotide is “derived from” a second polynucleotide if it has the same or substantially the same nucleotide sequence as a region of the second polynucleotide, its cDNA, complements thereof, or if it displays sequence identity as described above. This term is not meant to require or imply the polynucleotide must be obtained from the origin cited (although such is encompassed), but rather can be made by any suitable method.

A first polypeptide (or peptide) is “derived from” a second polypeptide (or peptide) if it is (i) encoded by a first polynucleotide derived from a second polynucleotide, or (ii) displays sequence identity to the second polypeptides as described above. This term is not meant to require or imply the polypeptide must be obtained from the origin cited (although such is encompassed), but rather can be made by any suitable method.

The term “in combination with” as used herein refers to uses where, for example, a first therapy is administered during the entire course of administration of a second therapy; where the first therapy is administered for a period of time that is overlapping with the administration of the second therapy, e.g., where administration of the first therapy begins before the administration of the second therapy and the administration of the first therapy ends before the administration of the second therapy ends; where the administration of the second therapy begins before the administration of the first therapy and the administration of the second therapy ends before the administration of the first therapy ends; where the administration of the first therapy begins before administration of the second therapy begins and the administration of the second therapy ends before the administration of the first therapy ends; where the administration of the second therapy begins before administration of the first therapy begins and the administration of the first therapy ends before the administration of the second therapy ends. As such, “in combination” can also refer to regimen involving administration of two or more therapies. “In combination with” as used herein also refers to administration of two or more therapies which can be administered in the same or different formulations, by the same or different routes, and in the same or different dosage form type.

The terms “treatment”, “treating”, “treat”, and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment”, as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting its development or progression; and (c) relieving the disease, i.e., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of a penetrating peptide composition that can elicit an immune response or confer immunity in the absence of a disease condition, e.g., in the case of a vaccine.

“Subject”, “host”, and “patient” are used interchangeably herein, to refer to an animal, human or non-human, amenable to therapy according to the methods of the disclosure or to which a peptide composition according to the present disclosure can be administered to achieve a desired effect. Generally, the subject is a mammalian subject.

The term “dermatitis”, as used herein, refers to inflammation of the skin and includes, for example, allergic contact dermatitis, urticaria, asteatotic dermatitis (dry skin on the lower legs), atopic dermatitis, contact dermatitis including irritant contact dermatitis and urushiol-induced contact dermatitis, eczema, gravitational dermatitis, nummular dermatitis, otitis externa, perioral dermatitis, and seborrhoeic dermatitis.

The term “stratum corneum” refers to the horny outer layer of the epidermis, consisting of several layers of flat, keratinized, non-nucleated, dead, or peeling cells.

As used in the claims, the term “comprising”, which is synonymous with “including”, “containing”, and “characterized by”, is inclusive or open-ended and does not exclude additional, unrecited elements and/or method steps. “Comprising” is a term of art that indicates that the named elements and/or steps are present, but that other elements and/or steps can be added and still fall within the scope of the relevant subject matter.

As used herein, the phrase “consisting of” excludes any element, step, and/or ingredient not specifically recited. For example, when the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, the peptides of the presently disclosed subject matter in some embodiments can “consist essentially of” a core amino acid sequence, which indicates that the peptide can include one or more (e.g., 1, 2, 3, 4, 5, 6, or more) N-terminal and/or C-terminal amino acids the presence of which does not materially affect the desired biological activity of the peptide.

With respect to the terms “comprising”, “consisting essentially of”, and “consisting of”, where one of these three terms is used herein, the presently disclosed subject matter can include the use of either of the other two terms. For example, the presently disclosed subject matter relates in some embodiments to compositions comprising the amino acid sequence TGSTQHQ (SEQ ID NO: 1). It is understood that the presently disclosed subject matter thus also encompasses peptides that in some embodiments consist essentially of the amino acid sequence TGSTQHQ (SEQ ID NO: 1); as well as peptides that in some embodiments consist of the amino acid sequence TGSTQHQ (SEQ ID NO: 1). Similarly, it is also understood that the methods of the presently disclosed subject matter in some embodiments comprise the steps that are disclosed herein and/or that are recited in the claims, that they in some embodiments consist essentially of the steps that are disclosed herein and/or that are recited in the claims, and that they in some embodiments consist of the steps that are disclosed herein and/or that are recited in the claim.

### II. Penetrating Peptides

The present disclosure provides peptides that are capable of penetrating the SC and/or penetrating viable cells following administration. These peptides are referred to herein as “penetrating peptides” or “SPACE Peptides”. In some embodiments, these penetrating peptides are capable of penetrating the cellular membranes of viable epidermal and dermal cells. Penetrating peptides according to the present disclosure can include, for example, one or more of the amino acid sequences provided in Table 2 below.

TABLE 2
Summary of Exemplary SPACE Peptide Sequences

| | | |
|---|---|---|
| TGSTQHQ | CTGSTQHQC | ACTGSTQHQCG |
| (SEQ ID | (SEQ ID | (SEQ ID |
| NO: 1) | NO: 7) | NO: 13) |
| HSALTKH | CHSALTKHC | ACHSALTKHCG |
| (SEQ ID | (SEQ ID | (SEQ ID |
| NO: 2) | NO: 8) | NO: 14) |
| KTGSHNQ | CKTGSHNQC | ACKTGSHNQCG |
| (SEQ ID | (SEQ ID | (SEQ ID |
| NO: 3) | NO: 9) | NO: 15) |
| MGPSSML | CMGPSSMLC | ACMGPSSMLCG |
| (SEQ ID | (SEQ ID | (SEQ ID |
| NO: 4) | NO: 10) | NO: 16) |
| TDPNQLQ | CTDPNQLQC | ACTDPNQLQCG |
| (SEQ ID | (SEQ ID | (SEQ ID |
| NO: 5) | NO: 11) | NO: 17) |
| STHFIDT | CSTHFIDTC | ACSTHFIDTCG |
| (SEQ ID | (SEQ ID | (SEQ ID |
| NO: 6) | NO: 12) | NO: 18) |

In some embodiments, penetrating peptides according to the present disclosure include an amino acid sequence including a stretch of three, four, five, six, or seven consecutive amino acids selected from one of the following amino acid sequences TGSTQHQ (SEQ ID NO: 1), HSALTKH (SEQ ID NO: 2), KTGSHNQ (SEQ ID NO: 3), MGPSSML (SEQ ID NO: 4), TDPNQLQ (SEQ ID NO: 5) and STHFIDT (SEQ ID NO: 6).

In some embodiments, penetrating peptides according to the present disclosure have an amino acid sequence from 8 to 11, 12 to 15, or 16 to 19 amino acids in length, including an amino acid sequence selected from one of the following amino acid sequences TGSTQHQ (SEQ ID NO: 1), HSALTKH (SEQ ID NO: 2), KTGSHNQ (SEQ ID NO: 3), MGPSSML (SEQ ID NO: 4), TDPNQLQ (SEQ ID NO: 5) and STHFIDT (SEQ ID NO: 6). In some embodiments, penetrating peptides according to the present disclosure have an amino acid sequence of at least 10, at least 20, at least 30, at least 40, at least 50, at least 60, at least 70, at least 80, at least 90, or at least 100 amino acids.

In some embodiments, penetrating peptides according to the present disclosure can be circularized by any of a variety of known cross-linking methods. In some embodiments, a penetrating peptide according to the present disclosure can be provided in a circularized conformation (i.e., as a cyclic peptide) in which a Cys-Cys disulfide bond is present. In some embodiments, penetrating peptides according to the present disclosure have an amino acid sequence including an internal amino acid sequence selected from one of the following amino acid sequences TGSTQHQ (SEQ ID NO: 1), HSALTKH (SEQ ID NO: 2), KTGSHNQ (SEQ ID NO: 3), MGPSSML (SEQ ID NO: 4), TDPNQLQ (SEQ ID NO: 5) and STHFIDT (SEQ ID NO: 6), wherein the amino acid sequence of the peptide includes at least a first Cys positioned external to the internal sequence in the N-terminal direction and at least a second Cys positioned external to the internal sequence in the C-terminal direction. Exemplary penetrating peptides according to the present disclosure that have an amino acid sequence including an internal amino acid sequence of one of SEQ ID NOs: 1-6 include, but are not limited to peptides comprising any of SEQ ID NOs: 7-18. In some embodiments, the two Cys residues present in any of SEQ ID NOs: 7-18 are employed to form a Cys-Cys disulfide bond. By way of example and not limitation, SEQ ID NO: 7 is the amino acid sequence CTGSTQHQC, which includes the internal sequence TGSTQHQ (SEQ ID NO: 1). In some embodiments, a cyclic penetrating peptide according to the present disclosure comprises a Cys-Cys disulfide bond between amino acid 1 and amino acid 9 of SEQ ID NO: 7. Similarly, SEQ ID NO: 8 is the amino acid sequence CHSALTKHC, which includes the internal sequence HSALTKH (SEQ ID NO: 2). In some embodiments, a cyclic penetrating peptide according to the present disclosure comprises a Cys-Cys disulfide bond between amino acid 1 and amino acid 9 of SEQ ID NO: 8. Also similarly, SEQ ID NO: 13 is the amino acid sequence ACTGSTQHQCG, which also includes the internal sequence TGSTQHQ (SEQ ID NO: 1). In some embodiments, a cyclic penetrating peptide according to the present disclosure comprises a Cys-Cys disulfide bond between amino acid 2 and amino acid 10 of SEQ ID NO: 13. As a final, non-limiting example, SEQ ID NO: 14 is the amino acid sequence ACHSALTKHCG, which also includes the internal sequence HSALTKH (SEQ ID NO: 2). In some embodiments, a cyclic penetrating peptide according to the present disclosure comprises a Cys-Cys disulfide bond between amino acid 2 and amino acid 10 of SEQ ID NO: 14.

In some embodiments, penetrating peptides according to the present disclosure include an amino acid sequence including an internal stretch of three, four, five, or six consecutive amino acids selected from one of the following amino acid sequences TGSTQHQ (SEQ ID NO: 1), HSALTKH (SEQ ID NO: 2), KTGSHNQ (SEQ ID NO: 3), MGPSSML (SEQ ID NO: 4), TDPNQLQ (SEQ ID NO: 5) and STHFIDT (SEQ ID NO: 6); and further including at least a first Cys positioned external to the internal sequence in the N-terminal direction and at least a second Cys positioned external to the internal sequence in the C-terminal direction.

The penetrating peptides disclosed herein include those having the amino acid sequences provided, as well as peptides having one or more amino acid substitutions, e.g., one or more conservative amino acid substitutions, relative to the sequences provided, wherein the peptides retains the capability of penetrating the SC or penetrating a cell.

### III. Active Agents

The ability of the above peptides to penetrate the SC following topical administration and/or to penetrate the cellular membranes of viable cells, e.g., epidermal and dermal cells, while conjugated to or associated with a molecular cargo, e.g., a low molecular weight compound or macromolecule, makes them suitable for facilitating the delivery of a wide variety of active agents known in the art.

General classes of active agents which can be delivered include, for example, proteins, peptides, nucleic acids, nucleotides, nucleosides and analogues thereof; as well as pharmaceutical compounds, e.g., low molecular weight compounds.

Active agents which can be delivered using the penetrating peptides disclosed herein include agents which act on the peripheral nerves, adrenergic receptors, cholinergic receptors, the skeletal muscles, the cardiovascular system, smooth muscles, the blood circulatory system, synaptic sites, neuroeffector junction sites, endocrine and hormone systems, the immunological system, the reproductive system, the skeletal system, autacoid systems, the alimentary and excretory systems, the histamine system and the central nervous system.

Suitable active agents can be selected, for example, from dermatological agents, anti-neoplastic agents, cardiovascular agents, renal agents, gastrointestinal agents, rheumatologic agents, immunological agents, and neurological agents among others.

Suitable dermatological agents can include, for example, local anesthetics, anti-inflammatory agents, anti-infective agents, agents to treat acne, anti-virals, anti-fungals, and agents for psoriasis such as topical corticosteroids, among others.

In some embodiments, a suitable dermatological agent is selected from the following list: 16-17A-Epoxyprogesterone (CAS Registry No. 1097-51-4), P-methoxycinnamic acid/4-Methoxycinnamic acid (CAS Registry No. 830-09-1), Octyl Methoxycinnamate (CAS Registry No. 5466-77-3), Octyl Methoxycinnamate (CAS Registry No. 5466-77-3), Methyl p-methoxy cinnamate (CAS Registry No. 832-01-9), 4-ESTREN-17β-OL-3-ONE (CAS Registry No. 62-90-8), Ethyl-p-anisoyl acetate (CAS Registry No. 2881-83-6), Dihydrouracil (CAS Registry No. 1904-98-9), Lopinavir (CAS Registry No. 192725-17-0), RITANSERIN(CAS Registry No. 87051-43-2), Nilotinib (CAS Registry No. 641571-10-0); Rocuronium bromide (CAS Registry No. 119302-91-9), p-Nitrobenzyl-6-(1-hydroxyethyl)-1-azabicyclo(3.2.0)heptane-3,7-dione-2-carboxylate (CAS Registry No. 74288-40-7), Abamectin (CAS Registry No. 71751-41-2), Paliperidone (CAS Registry No. 144598-75-4), Gemifioxacin (CAS Registry No. 175463-14-6), Valrubicin (CAS Registry No. 56124-62-0), Mizoribine (CAS Registry No. 50924-49-7), Solifenacin succinate (CAS Registry No. 242478-38-2), Lapatinib (CAS Registry No. 231277-92-2), Dydrogesterone (CAS Registry No. 152-62-5), 2,2-Dichloro-N-[(1R,2S)-3-fluoro-1-hydroxy-1-(4-methylsulfonylphenyl)propan-2-yl]acetamide (CAS Registry No. 73231-34-2), Tilmicosin (CAS Registry No. 108050-54-0), Efavirenz (CAS Registry No. 154598-52-4), Pirarubicin (CAS Registry No. 72496-41-4), Nateglinide (CAS Registry No. 105816-04-4), Epirubicin (CAS Registry No. 56420-45-2), Entecavir (CAS Registry No. 142217-69-4), Etoricoxib (CAS Registry No. 202409-33-4), Cilnidipine (CAS Registry No. 132203-70-4), Doxorubicin hydrochloride (CAS Registry No. 25316-40-9), Escitalopram (CAS Registry No. 128196-01-0), Sitagliptin phosphate monohydrate (CAS Registry No. 654671-77-9), Acitretin (CAS Registry No. 55079-83-9), Rizatriptan benzoate (CAS Registry No. 145202-66-0), Doripenem (CAS Registry No. 148016-81-3), Atracurium besylate (CAS Registry No. 64228-81-5), Nilutamide (CAS Registry No. 63612-50-0), 3,4-Dihydroxyphenylethanol (CAS Registry No. 10597-60-1), KETANSERIN TARTRATE (CAS Registry No. 83846-83-7), Ozagrel (CAS Registry No. 82571-53-7), Eprosartan mesylate (CAS Registry No. 144143-96-4), Ranitidine hydrochloride (CAS Registry No. 66357-35-5), 6,7-Dihydro-6-mercapto-5H-pyrazolo[1,2-a][1,2,4]triazolium chloride (CAS Registry No. 153851-71-9), Sulfapyridine (CAS Registry No. 144-83-2), Teicoplanin (CAS Registry No. 61036-62-2), Tacrolimus (CAS Registry No. 104987-11-3), LUMIRACOXIB (CAS Registry No. 220991-20-8), Allyl alcohol (CAS Registry No. 107-18-6), Protected meropenem (CAS Registry No. 96036-02-1), Nelarabine (CAS Registry No. 121032-29-9), Pimecrolimus (CAS Registry No. 137071-32-0), 4-[6-Methoxy-7-(3-piperidin-1-ylpropoxy)quinazolin-4-yl]-N-(4-propan-2-yloxyphenyl)piperazine-1-carboxamide (CAS Registry No. 387867-13-2), Ritonavir (CAS Registry No. 155213-67-5), Adapalene (CAS Registry No. 106685-40-9), Aprepitant (CAS Registry No. 170729-80-3), Eplerenone (CAS Registry No. 107724-20-9), Rasagiline mesylate (CAS Registry No. 161735-79-1), Miltefosine (CAS Registry No. 58066-85-6), Raltegravir potassium (CAS Registry No. 871038-72-1), Dasatinib monohydrate (CAS Registry No. 863127-77-9), OXOMEMAZINE (CAS Registry No. 3689-50-7), Pramipexole (CAS Registry No. 104632-26-0), PARECOXIB SODIUM (CAS Registry No. 198470-85-8), Tigecycline (CAS Registry No. 220620-09-7), Toltrazuril (CAS Registry No. 69004-03-1), Vinflunine (CAS Registry No. 162652-95-1), Drospirenone (CAS Registry No. 67392-87-4), Daptomycin (CAS Registry No. 103060-53-3), Montelukast sodium (CAS Registry No. 151767-02-1), Brinzolamide (CAS Registry No. 138890-62-7), Maraviroc (CAS Registry No. 376348-65-1), Doxercalciferol (CAS Registry No. 54573-75-0), Oxolinic acid (CAS Registry No. 14698-29-4), Daunorubicin hydrochloride (CAS Registry No. 23541-50-6), Nizatidine (CAS Registry No. 76963-41-2), Idarubicin (CAS Registry No. 58957-92-9), FLUOXETINE HYDROCHLORIDE (CAS Registry No. 59333-67-4), Ascomycin (CAS Registry No. 11011-38-4), beta-Methyl vinyl phosphate (MAP) (CAS Registry No. 90776-59-3), Amorolfine (CAS Registry No. 67467-83-8), Fexofenadine HCl (CAS Registry No. 83799-24-0), Ketoconazole (CAS Registry No. 65277-42-1), 9,10-difluoro-2,3-dihydro-3-me-7-oxo-7H-pyrido-1 (CAS Registry No. 82419-35-0), Ketoconazole (CAS Registry No. 65277-42-1), Terbinafine HCl (CAS Registry No. 78628-80-5), Amorolfine (CAS Registry No. 78613-35-1), Methoxsalen (CAS Registry No. 298-81-7), Olopatadine HCl (CAS Registry No. 113806-05-6), Zinc Pyrithione (CAS Registry No. 13463-41-7), Olopatadine HCl (CAS Registry No. 140462-76-6), Cyclosporin (CAS Registry No. 59865-13-3), Hyaluronic acid (CAS Registry No. 9004-61-9), and Botulinum toxin and its analogs and vaccine components.

III.A. Protein, Polypeptides, and Peptides as Active Agents

Proteins useful in the disclosed depot formulations can include, for example, molecules such as cytokines and their receptors, as well as chimeric proteins including cytokines or their receptors, including, for example tumor necrosis factor alpha and beta, their receptors and their derivatives; renin; growth hormones, including human growth hormone, bovine growth hormone, methionine-human growth hormone, des-phenylalanine human growth hormone, and porcine growth hormone; growth hormone releasing factor (GRF); parathyroid and pituitary hormones; thyroid stimulating hormone; human pancreas hormone releasing factor; lipoproteins; colchicine; prolactin; corticotrophin; thyrotropic hormone; oxytocin; vasopressin; somatostatin; lypressin; pancreozymin; leuprolide; alpha-1-antitrypsin; insulin A-chain; insulin B-chain; proinsulin; follicle stimulating hormone; calcitonin; luteinizing hormone; luteinizing hormone releasing hormone (LHRH); LHRH agonists and antagonists; glucagon; clotting factors such as factor VIIIC, factor IX, tissue factor, and von Willebrand factor; anti-clotting factors such as Protein C; atrial natriuretic factor; lung surfactant; a plasminogen activator other than a tissue-type plasminogen activator (t-PA), for example a urokinase; bombesin; thrombin; hematopoietic growth factor; enkephalinase; RANTES (regulated on activation normally T-cell expressed and secreted); human macrophage inflammatory protein (MIP-1-alpha); a serum albumin such as human serum albumin; mullerian-inhibiting substance; relaxin A-chain; relaxin B-chain; prorelaxin; mouse gonadotropin-associated peptide; chorionic gonadotropin; gonadotropin releasing hormone; bovine somatotropin; porcine somatotropin; a microbial protein, such as beta-lactamase; DNase; inhibin; activin; vascular endothelial growth factor (VEGF); receptors for hormones or growth factors; integrin; protein A or D; rheumatoid factors; a neurotrophic factor such as bone-derived neurotrophic factor (BDNF), neurotrophin-3, 4, -5, or -6 (NT-3, NT-4, NT-5, or NT-6), or a nerve growth factor such as NGF-β; platelet-derived growth factor (PDGF); fibroblast growth factor such as acidic FGF and basic FGF; epidermal growth factor (EGF); transforming growth factor (TGF) such as TGF-alpha and TGF-beta, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-19; erythropoietin; osteoinductive factors; immunotoxins; a bone morphogenetic protein (BMP); an interferon such as interferon-alpha (e.g., interferon α2A), -beta, -gamma, -lambda, and consensus interferon; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the HIV-1 envelope glycoprotein, gp120, gp160 or fragments thereof; transport proteins; homing receptors; addressins; fertility inhibitors such as the prostaglandins; fertility promoters; regulatory proteins; antibodies (including fragments thereof) and chimeric proteins, such as immunoadhesins; precursors, derivatives, prodrugs and analogues of these compounds, and pharmaceutically acceptable salts of these compounds, or their precursors, derivatives, prodrugs and analogues.

Suitable proteins or peptides can be native or recombinant and include, e.g., fusion proteins.

In some embodiments, the protein is a growth hormone, such as human growth hormone (hGH), recombinant human growth hormone (rhGH), bovine growth hormone, methionine-human growth hormone, des-phenylalanine human growth hormone, and porcine growth hormone; insulin, insulin A-chain, insulin B-chain, and proinsulin; or a growth factor, such as vascular endothelial growth factor (VEGF), nerve growth factor (NGF), platelet-derived growth factor (PDGF), fibroblast growth factor (FGF), epidermal growth factor (EGF), transforming growth factor (TGF), and insulin-like growth factor-I and -II (IGF-I and IGF-II).

Suitable peptides for use as the active agent in the injectable, biodegradable delivery depots disclosed herein include, but are not limited to, Glucagon-like peptide-1 (GLP-1) and precursors, derivatives, prodrugs and analogues thereof.

III.B. Nucleic Acids as Active Agents

Nucleic acid active agents include nucleic acids as well as precursors, derivatives, prodrugs and analogues thereof, e.g., therapeutic nucleotides, nucleosides and analogues thereof; therapeutic oligonucleotides; and therapeutic polynucleotides. Active agents selected from this group can find particular use as anticancer agents and antivirals. Suitable nucleic acid active agents can include for example ribozymes, antisense oligodeoxynucleotides, aptamers and siRNA. Examples of suitable nucleoside analogues include, but are not limited to, cytarabine (araCTP), gemcitabine (dFdCTP), and floxuridine (FdUTP). In some embodiments, a suitable nucleic acid active agent is an interfering RNA, e.g., shRNA, miRNA or siRNA. Suitable siRNAs include, for example, IL-7 (Interleukin-7) siRNA, IL-10 (Interleukin-10) siRNA, IL-22 (Interleukin-22) siRNA, IL-23 (Interleukin 23) siRNA, CD86 siRNA, KRT6a (keratin 6A) siRNA, K6a N171K (keratin 6a N171K) siRNA, TNFα (tumor necrosis factor α) siRNA, TNFR1 (tumor necrosis factor receptor-1) siRNA, TACE (tumor necrosis factor (TNF)-α converting enzyme) siRNA, RRM2 (ribonucleotide reductase subunit-2) siRNA, and VEGF (vascular endothelial growth factor) siRNA. mRNA sequences of the human gene targets of these siRNAs are known in the art. For IL-7, see e.g., GENBANK® Accession No. NM_{—}000880.3, GENBANK® Accession No. NM_{—}001199886.1, GENBANK® Accession No. NM_{—}001199887.1, and GENBANK® Accession No. NM_{—}001199888.1; for IL-10, see e.g., GENBANK® Accession No. NM_{—}000572.2; for IL-22 see e.g., GENBANK® Accession No. NM_{—}020525.4; for IL-23, see e.g., GENBANK® Accession No. NM_{—}016584.2, and GENBANK® Accession No. AF301620.1; for CD86, see e.g., GENBANK® Accession No. NM_{—}175862.4, GENBANK® Accession No. NM_{—}006889.4, GENBANK® Accession No. NM_{—}176892.1, GENBANK® Accession No. NM_{—}001206924.1, and GENBANK® Accession No. NM_{—}001206925.1; for KRT6a, see e.g., GENBANK® Accession No. NM_{—}005554.3; for TNFα, see e.g., GENBANK® Accession No. NM_{—}000594.2; for TNFR1, see e.g., GENBANK® Accession No. NM_{—}001065.3; for TACE, see e.g., GENBANK® Accession No. NM_{—}003183.4; for RRM2, see e.g., GENBANK® Accession No. NM_{—}001165931.1 and GENBANK® Accession No. NM_{—}001034.3; for VEGF, see e.g., GENBANK® Accession No. NM_{—}001025366.2, GENBANK® Accession No. NM_{—}001025367.2, GENBANK® Accession No. NM_{—}001025368.2, GENBANK® Accession No. NM_{—}001025369.2, GENBANK® Accession No. NM_{—}001025370.2, NM_{—}001033756.2, GENBANK® Accession No. NM_{—}001171622.1, and GENBANK® Accession No. NM_{—}003376.5.

In addition a variety of methods and techniques are known in the art for selecting a particular mRNA target sequence during siRNA design. See e.g., the publicly available siRNA design tool provided by the Whitehead Institute of Biomedical Research at MIT. This tool can be located on the internet on the website located by placing http:// directly preceding jura.wi.mit.edu/bioc/siRNAext/.

III.C. Additional Active Agent Compounds

A variety of additional active agent compounds can be used in the injectable depot compositions disclosed herein. Suitable compounds can include compounds directed to one or more of the following drug targets: Kringle domain, Carboxypeptidase, Carboxylic ester hydrolases, Glycosylases, Rhodopsin-like dopamine receptors, Rhodopsin-like adrenoceptors, Rhodopsin-like histamine receptors, Rhodopsin-like serotonin receptors, Rhodopsin-like short peptide receptors, Rhodopsin-like acetylcholine receptors, Rhodopsin-like nucleotide-like receptors, Rhodopsin-like lipid-like ligand receptors, Rhodopsin-like melatonin receptors, Metalloprotease, Transporter ATPase, Carboxylic ester hydrolases, Peroxidase, Lipoxygenase, DOPA decarboxylase, A/G cyclase, Methyltransferases, Sulphonylurea receptors, other transporters (e.g., Dopamine transporter, GABA transporter 1, Norepinephrine transporter, Potassium-transporting ATPase α-chain 1, Sodium-(potassium)-chloride cotransporter 2, Serotonin transporter, Synaptic vesicular amine transporter, and Thiazide-sensitive sodium-chloride cotransporter), Electrochemical nucleoside transporter, Voltage-gated ion channels, GABA receptors (Cys-Loop), Acetylcholine receptors (Cys-Loop), NMDA receptors, 5-HT3 receptors (Cys-Loop), Ligand-gated ion channels Glu: kainite, AMPA Glu receptors, Acid-sensing ion channels aldosterone, Ryanodine receptors, Vitamin K epoxide reductase, MetGluR-like GABA_{B} receptors, Inwardly rectifying K⁺ channel, NPC1L1, MetGluR-like calcium-sensing receptors, Aldehyde dehydrogenases, Tyrosine 3-hydroxylase, Aldose reductase, Xanthine dehydrogenase, Ribonucleoside reductase, Dihydrofolate reductase, IMP dehydrogenase, Thioredoxin reductase, Dioxygenase, Inositol monophosphatase, Phosphodiesterases, Adenosine deaminase, Peptidylprolyl isomerases, Thymidylate synthase, Aminotransferases, Farnesyl diphosphate synthase, Protein kinases, Carbonic anhydrase, Tubulins, Troponin, Inhibitor of IκB kinase-β, Amine oxidases, Cyclooxygenases, Cytochrome P450s, Thyroxine 5-deiodinase, Steroid dehydrogenase, HMG-CoA reductase, Steroid reductases, Dihydroorotate oxidase, Epoxide hydrolase, Transporter ATPase, Translocator, Glycosyltransferases, Nuclear receptors NR3 receptors, Nuclear receptors: NR1 receptors, and Topoisomerase.

In some embodiments, the active agent is a compound targeting one of rhodopsin-like GPCRs, nuclear receptors, ligand-gated ion channels, voltage-gated ion channels, penicillin-binding protein, myeloperoxidase-like, sodium: neurotransmitter symporter family, type II DNA topoisomerase, fibronectin type III, and cytochrome P450.

In some embodiments, the active agent is an anticancer agent. Suitable anticancer agents include, but are not limited to, Actinomycin D, Alemtuzumab, Allopurinol sodium, Amifostine, Amsacrine, Anastrozole, Ara-CMP, Asparaginase, Azacytadine, Bendamustine, Bevacizumab, Bicalutimide, Bleomycin (e.g., Bleomycin A₂ and B₂), Bortezomib, Busulfan, Camptothecin sodium salt, Capecitabine, Carboplatin, Carmustine, Cetuximab, Chlorambucil, Cisplatin, Cladribine, Clofarabine, Cyclophosphamide, Cytarabine, Dacarbazine, Dactinomycin, Daunorubicin, Daunorubicin liposomal, Dacarbazine, Decitabine, Docetaxel, Doxorubicin, Doxorubicin liposomal, Epirubicin, Estramustine, Etoposide, Etoposide phosphate, Exemestane, Floxuridine, Fludarabine, Fludarabine phosphate, 5-Fluorouracil, Fotemustine, Fulvestrant, Gemcitabine, Goserelin, Hexamethylmelamine, Hydroxyurea, Idarubicin, Ifosfamide, Imatinib, Irinotecan, Ixabepilone, Lapatinib, Letrozole, Leuprolide acetate, Lomustine, Mechlorethamine, Melphalan, 6-Mercaptopurine, Methotrexate, Mithramycin, Mitomycin C, Mitotane, Mitoxantrone, Nimustine, Ofatumumab, Oxaliplatin, Paclitaxel, Panitumumab, Pegaspargase, Pemetrexed, Pentostatin, Pertuzumab, Picoplatin, Pipobroman, Plerixafor, Procarbazine, Raltitrexed, Rituximab, Streptozocin, Temozolomide, Teniposide, 6-Thioguanine, Thiotepa, Topotecan, Trastuzumab, Treosulfan, Triethylenemelamine, Trimetrexate, Uracil Nitrogen Mustard, Valrubicin, Vinblastine, Vincristine, Vindesine, Vinorelbine, and analogues, precursors, derivatives and pro-drugs thereof. It should be noted that two or more of the above compounds can be used in combination in the penetrating peptide compositions of the present disclosure.

Active agents of interest for use in the disclosed penetrating peptide compositions can also include opioids and derivatives thereof as well as opioid receptor agonists and antagonists, e.g., naltrexone, naloxone, nalbuphine, fentanyl, sufentanil, oxycodone, and pharmaceutically acceptable salts and derivatives thereof.

In some embodiments the active agent is a small molecule or low molecular weight compound, e.g., a molecule or compound having a molecular weight of less than or equal to about 1000 Daltons, e.g., less than or equal to about 800 Daltons.

In some embodiments, the active agent is a label. Suitable labels include, e.g., radioactive isotopes, fluorescers, chemiluminescers, chromophores, enzymes, enzyme substrates, enzyme cofactors, enzyme inhibitors, chromophores, dyes, metal ions, magnetic particles, nanoparticles and quantum dots.

The active agent can be present in any suitable concentration in the compositions disclosed herein. Suitable concentrations can vary depending on the potency of the active agent, active agent half-life, etc. In addition, penetrating peptide compositions according to the present disclosure can include one or more active agents, e.g., a combination of two or more of the active agents described above.

### IV. Active Agent Carriers

As described previously herein one or more active agents can be conjugated to or associated with a penetrating peptide to provide a penetrating peptide composition according to the present disclosure. Alternatively, a penetrating peptide composition according to the present disclosure can include a penetrating peptide as disclosed herein conjugated or associated with an active agent carrier (also referred to herein as a “carrier”) which in turn includes the active agent attached thereto and/or disposed therein.

Suitable active agent carriers include, for example, liposomes, nanoparticles, micelles, microbubbles, and the like. Techniques for incorporating active agents into such carriers are known in the art. For example, liposomes or lipidic particles can be prepared in accordance with U.S. Pat. No. 5,077,057 to Szoka, Jr. Liposomes formed from nonphosphal lipid components which have the potential to form lipid bilayers are disclosed in Biochim. Biophys. Acta, 19: 227-232 (1982). For the preparation, purification, modification and loading of liposomes see generally, New, R.C.C., Liposomes: A Practical Approach, (1990) Oxford University Press Inc., N.Y.

A general discussion of techniques for preparation of liposomes and of medication encapsulating liposomes can be found in U.S. Pat. No. 4,224,179 to Schneider. See also Mayer et al., Chemistry and Physics of Lipids, 40: 333-345 (1986). See also U.S. Pat. No. 6,083,529 to Manzo et al. for the encapsulation of an active agent dry powder composition. For incorporation of active agents into nanoparticles, see e.g., M. M. de Villiers et al. (editors), Nanotechnology in Drug Delivery, (2009) American Associate of Pharmaceutical Scientists, Springer: AAPS Press, New York, N.Y. For incorporation of active agents into micelles, see e.g., D. R. Lu and S. Oie, Cellular Drug Delivery: Principles and Practice, (2004) Humana Press Inc., Totowa, N.J.

In some embodiments, an active agent carrier of the presently disclosed subject matter is an ethosome. Ethosomes are vesicles formed typically from phospholipids in the presence of water and ethanol or another alcohol, and sometimes further in the presence of glycols and other polyols. Ethosomes can be prepared by techniques that would be known to one of ordinary skill in the art, and are set forth in, for example, U.S. Pat. Nos. 5,540,934 and 5,716,638, both to Touitou; and in Godin and Touitou (2003) Crit. Rev Ther Drug Carrier Syst 20:63-102. In some embodiments, an SPACE Peptide-containing ethosome of the presently disclosed subject matter is prepared by mixing lipids and/or phospholipids, particularly includes at least one functionalized lipid and/or phospholipid with one or more SPACE Peptides in a volume of water that in some embodiments can contain ethanol and/or sodium phosphate buffer. In some embodiments, CsA, HA, or any other bioactive agent can also be added to the mixture to allow SPACE Peptide-containing micelles, liposomes, and/or ethosomes for form, which encapsulate the CsA, HA, or other bioactive agent. In some embodiments, a SPACE Peptide (in some embodiments, the same SPACE Peptide as used during the micelle/liposome/ethosome formation step, in some embodiments a different SPACE Peptide as used during the micelle/liposome/ethosome formation step, and in some embodiments a mixture of the same and/or one or more different SPACE Peptides as used during the micelle/liposome/ethosome formation step) is added after micelle/liposome/ethosome formation to produce a composition comprising a SPACE Peptide-containing micelle/liposome/ethosome that also comprises one or more free SPACE Peptides.

In some embodiments, an active agent carrier of the presently disclosed subject matter is a micelle, liposome, and/or ethosome comprising one or more SPACE Peptides conjugated with an alkyl chain. Methods for preparing alkyl-conjugated peptides include but are not limited to those disclosed in, for example, Peters et al. (2009) PNAS Vol. 106, No. 24: 9815-9819.

### V. Attachment of Peptides to Active Agents and Active Agent Carriers

Penetrating peptides as described herein can be conjugated to or associated with an active agent. Alternatively, a penetrating peptide as disclosed herein can be conjugated or associated with an active agent carrier, which in turn includes the active agent attached thereto and/or disposed therein (examples of which are discussed above). Conjugation techniques generally result in the formation of one or more covalent bonds between the penetrating peptide and either the active agent or an active agent carrier while association techniques generally utilize one or more of hydrophobic, electrostatic or van der Walls interactions.

A variety of techniques can be used for conjugating or associating a peptide to an active agent. Similarly, a variety of techniques can be used for conjugating or associating a peptide to an active agent carrier, e.g., liposomes, nanoparticles, or micelle as described herein.

For example, where the active agent is a peptide or polypeptide, the entire composition, including the penetrating peptide, can be synthesized using standard amino acid synthesis techniques. Other methods including standard molecular biology techniques can be used to express and purify the entire polypeptide sequence including the penetrating peptide. Additional methods of conjugating peptides to other peptides or polypeptides include Cu-catalyzed azide/alkyne [3+2] cycloaddition “Click Chemistry” as described by Rostovtsev et al. (2002) Angew. Chem. Int. Ed. 41: 2596-2599 and Tornoe et al. (2002) J. Org. Chem. 67: 3057-3064; azide/DIFO (Difluorinated Cyclooctyne) Cu-free Click Chemistry as described by Baskin et al. (2007) PNAS Vol. 104, No. 43: 167393-16797; azide/phosphine “Staudinger Reaction” as described by Lin et al. (2005) J. Am. Chem. Soc. 127: 2686-2695; azide/triarylphosphine “Modified Staudinger Reaction” as described by Saxon and Bertozzi (2000) March 17 Science 287(5460): 2007-10; and catalyzed olefin cross metathesis reactions as described by Casey (2006) J. of Chem. Edu. Vol. 83, No. 2: 192-195, Lynn et al. (2000) J. Am. Chem. Soc. 122: 6601-6609, and Chen et al. (2003) Progress in Chemistry 15: 401-408.

Where the active agent is a low molecular weight compound or small molecule, a variety of techniques can be utilized to conjugate the low molecular weight compound or small molecule to a penetrating peptide as described herein, e.g., Click chemistry as described in Loh et al., Chem Commun (Camb), 2010 Nov. 28; 46(44): 8407-9. Epub 2010 Oct. 7. See also, Thomson S., Methods Mol. Med. (2004) 94: 255-265, describing conjugation of small molecule carboxyl, hydroxyl, and amine residues to amine and sulfhydryl residues on proteins.

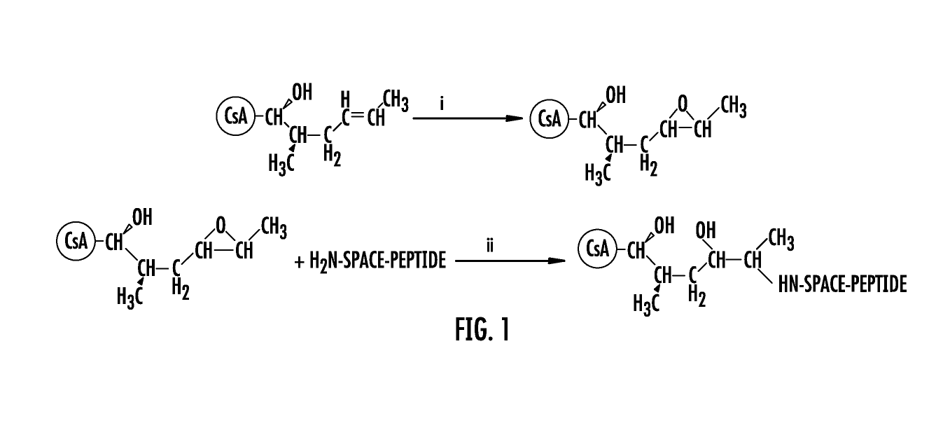

By way of example and not limitation, a SPACE Peptide can be conjugated to cyclosporin A (CsA) as set forth in the method of FIG. 1. Briefly, an epoxide derivative of CsA is prepared, and reacted with a SPACE Peptide under conditions wherein the N-terminal amino group of the SPACE Peptide attacks the epoxide ring to produce a CsA-SPACE Peptide conjugate. The CsA-SPACE Peptide conjugate can then be employed as set forth herein, including as is, associated with an active agent carrier, encapsulated by an active agent carrier, etc.

Methods are also available in the art for conjugating peptides to active agent carriers such as liposomes. See e.g., G. Gregoriadis (editor), Liposome Technology Third Edition, Volume II Entrapment of Drugs and Other materials into Liposomes, (2007), Informa Healthcare, New York, N.Y., which describes techniques for coupling peptides to the surface of liposomes. For the covalent attachment of proteins, to liposomes see New, R.C.C., Liposomes: A Practical Approach, (1990) Oxford University Press Inc., N.Y. at pages 163-182.

### VI. Administration of Penetrating Peptide Compositions as Pharmaceutical Formulations

One skilled in the art will appreciate that a variety of suitable methods of administering a penetrating peptide composition to a subject or host, e.g., patient, in need thereof, are available, and, although more than one route can be used to administer a particular composition, a particular route can provide a more immediate and more effective reaction than another route. Pharmaceutically acceptable excipients are also well known to those who are skilled in the art, and are readily available. The choice of excipient will be determined in part by the particular compound, as well as by the particular method used to administer the composition. Accordingly, there are a wide variety of suitable formulations of the penetrating peptide compositions. The following methods and excipients are merely exemplary and are in no way limiting.

Formulations suitable for oral administration can consist of (a) liquid solutions, such as an effective amount of the compound dissolved in diluents, such as water, saline, or orange juice; (b) capsules, sachets or tablets, each containing a predetermined amount of the active ingredient, as solids or granules; (c) suspensions in an appropriate liquid; (d) suitable emulsions and (e) hydrogels. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles including the active ingredient in an inert base, such as gelatin and glycerin, or sucrose and acacia, emulsions, gels, and the like containing, in addition to the active ingredient, such excipients as are known in the art.

Penetrating peptide formulations can be made into aerosol formulations to be administered via inhalation. These aerosol formulations can be placed into pressurized acceptable propellants, such as dichlorodifluoromethane, propane, nitrogen, and the like. They can also be formulated as pharmaceuticals for non-pressured preparations such as for use in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Formulations suitable for topical administration can be presented as creams, gels, pastes, patches, sprays or foams.

Suppository formulations are also provided by mixing with a variety of bases such as emulsifying bases or water-soluble bases. Formulations suitable for vaginal administration can be presented as pessaries, tampons, creams, gels, pastes, foams.

Unit dosage forms for oral or rectal administration such as syrups, elixirs, and suspensions can be provided wherein each dosage unit, for example, teaspoonful, tablespoonful, tablet or suppository, contains a predetermined amount of the composition. Similarly, unit dosage forms for injection or intravenous administration can comprise the penetrating peptides in a formulation as a solution in sterile water, normal saline or another pharmaceutically acceptable carrier.

The term “unit dosage form”, as used herein, refers to physically discrete units suitable as unitary dosages for human and animal subjects, each unit containing a predetermined quantity of penetrating peptide composition calculated in an amount sufficient to produce the desired effect in association with a pharmaceutically acceptable diluent, carrier or vehicle. The specifications for the novel unit dosage forms of the penetrating peptide compositions depend on the particular active agent employed and the effect to be achieved, and the pharmacodynamics associated with each compound in the host.

Those of skill in the art will readily appreciate that dose levels can vary as a function of the specific compound, the nature of the delivery vehicle, and the like. Suitable dosages for a given compound are readily determinable by those of skill in the art by a variety of standard methodologies.

Optionally, the pharmaceutical composition can contain other pharmaceutically acceptable components, such a buffers, surfactants, antioxidants, viscosity modifying agents, preservatives and the like. Each of these components is well-known in the art. See e.g., U.S. Pat. No. 5,985,310, the disclosure of which is herein incorporated by reference.

Other components suitable for use in penetrating peptide formulations can be found in Remington's Pharmaceutical Sciences, 18th edition, Mack Pub. Co., (June 1995). In an embodiment, the aqueous cyclodextrin solution further comprise dextrose, e.g., about 5% dextrose.

### VII. Administration of Penetrating Peptide Compositions as Medical Device Components

In some embodiments, one or more of the penetrating peptide compositions of the present disclosure can be incorporated into a medical device known in the art, for example, drug eluting stents, catheters, fabrics, cements, bandages (liquid or solid), biodegradable polymer depots and the like. In some embodiments, the medical device is an implantable or partially implantable medical device.

### VIII. Methods of Treatment

The terms “an effective amount” (or, in the context of a therapy, a “pharmaceutically effective amount”) of a penetrating peptide composition generally refers to an amount of the penetrating peptide composition, effective to accomplish the desired therapeutic effect, e.g., in the case of a penetrating peptide-siRNA composition, an amount effective to reduce expression of the targeted mRNA by an amount effective to produce a desired therapeutic effect.

Effective amounts of penetrating peptide compositions, suitable delivery vehicles, and protocols can be determined by conventional methodologies. For example, in the context of therapy a medical practitioner can commence treatment with a low dose of one or more penetrating peptide compositions in a subject or patient in need thereof, and then increase the dosage, or systematically vary the dosage regimen, monitor the effects thereof on the patient or subject, and adjust the dosage or treatment regimen to maximize the desired therapeutic effect. Further discussion of optimization of dosage and treatment regimens can be found in Benet et al., in Goodman and Gilman's The Pharmacological Basis of Therapeutics, Ninth Edition, Hardman et al., Eds., McGraw-Hill, N.Y., (1996), Chapter 1, pp. 3-27, and L. A. Bauer, in Pharmacotherapy, A Pathophysiologic Approach, Fourth Edition, DiPiro et al., Eds., Appleton & Lange, Stamford, Conn., (1999), Chapter 3, pp. 21-43, and the references cited therein, to which the reader is referred.

The dosage levels and mode of administration will be dependent on a variety of factors such as the penetrating peptides used, the active agent, the context of use (e.g., the patient to be treated), and the like. Optimization of modes of administration, dosage levels, and adjustment of protocols, including monitoring systems to assess effectiveness are routine matters well within ordinary skill.

In one embodiment, the present disclosure provides a method of treating a subject having a dermatological disease, including: administering to the subject a pharmaceutically effective amount of a composition including a penetrating peptide as disclosed herein, wherein the peptide is conjugated to or associated with a dermatological active agent, e.g., a dermatological active agent as disclosed herein, or a dermatological active agent carrier including the active agent.

In one embodiment, the present disclosure provides a method of treating a subject having, suspected of having or susceptible to a disorder resulting at least in part from expression of an mRNA, including administering to the subject a pharmaceutically effective amount of a composition including a penetrating peptide as described herein, wherein the penetrating peptide is conjugated to or associated with an interfering RNA or an active agent carrier including an interfering RNA, e.g., an shRNA, miRNA or siRNA which targets the mRNA or a carrier including the interfering RNA.

In one embodiment, the interfering RNA is an siRNA, e.g., an siRNA selected from one of the following: an IL-10 siRNA, an IL-14 siRNA, an IL-17 siRNA, an IL-22 siRNA, an IL-23 siRNA, a CD86 siRNA, a KRT6a siRNA, a TNFR1 siRNA, a TNFα siRNA, and a TACE siRNA. siRNAs can be designed to target mRNAs encoding other gene products with desired bioactivities including, but not limited to mRNAs encoding members of the keratin family, the collagen family, other cytokine families, growth factor families, adhesion protein families, angiogenesis-promoting protein families, etc.

### IX. Cosmetic Uses

In some embodiments, the compositions of the presently disclosed subject matter can be employed in a cosmetic formulation and/or for cosmetic uses. Thus, in some embodiments the compositions of the presently disclosed subject matter can be solubilized in a cosmetic carrier such as liposomes, or adsorbed on powdery organic polymers, mineral supports such as talcs and bentonites, and more generally solubilized in, or fixed on, any physiologically acceptable carrier.

In some embodiments, the composition of the presently disclosed subject matter can be applied by any appropriate route, notably oral, parenteral, or topical, and the formulation of the cosmetic compositions can be adapted by the person skilled in the art, in particular for cosmetic or dermatological compositions. In some embodiments, the compositions of the presently disclosed subject matter can be formulated for topical administration. These compositions therefore can contain a physiologically acceptable medium (i.e., a medium compatible with the skin and epithelial appendages) and cover all cosmetic or dermatological forms.

It is understood that the active agents of the presently disclosed subject matter can be used alone or in combination with other active principles.

The compositions of the presently disclosed subject matter can also contain various protective or anti-aging active principles intended to promote and supplement the action of the active agents. By way of example and not limitation, the following ingredients can be included, either alone or in combination: cicatrizant, anti-age, anti-wrinkle, smoothing, anti-radical, anti-UV agents, agents stimulating the synthesis of dermal macromolecules or energy metabolism, moisturizing, antibacterial, antifungal, anti-inflammatory, anesthetic agents, agents modulating cutaneous differentiation, pigmentation or depigmentation, agents stimulating nail or hair growth. Alternative or in addition, other active agents having an anti-radical or antioxidant action, chosen from among vitamin C, vitamin E, coenzyme Q10, polyphenolic plant extracts, and/or retinoids, can also be added.

In some embodiments, the compositions of the presently disclosed subject matter can also include other active agents that stimulate the synthesis of dermal macromolecules (laminin, fibronectin, collagen), for example the collagen peptide sold under the name COLLAXYL® by Vincience S A, Sophia Antipolis, France.

In some embodiments, cosmetic compositions of the presently disclosed subject matter can be present in the form of an aqueous solution, hydroalcoholic or oily solution; an oil in water emulsion, water in oil emulsion or multiple emulsions; creams, suspensions, powders, etc., that are suitable for application on the skin, mucosa, lips, and/or epithelial appendages. These compositions can be more or less fluid and in some embodiments have the appearance of a cream, a lotion, a milk, a serum, a pomade, a gel, a paste, or a foam. They can also be present in solid form, such as a stick, or can be applied on the skin in aerosol form. They can be used as a care product and/or as a skin makeup product.

All of the compositions also comprise any additive commonly used in the contemplated field of application as well as the adjuvants necessary for their formulation, such as solvents, thickeners, diluents, antioxidants, colorants, sunscreens, self-tanning agents, pigments, fillers, preservatives, fragrances, odor absorbers, other cosmetic active principles, essential oils, vitamins, essential fatty acids, surface active agents, film-forming polymers, etc. One of ordinary skill in the art can make sure that these adjuvants as well as their proportions are chosen so as to not harm the desired advantageous properties of the presently disclosed compositions. These adjuvants can, for example, correspond to a concentration ranging from 0.01 to 20% of the total weight of the composition. When the composition of the presently disclosed subject matter is an emulsion, the fatty phase can represent in some embodiments from 5 to 80% by weight and in some embodiments from 5 to 50% by weight with relation to the total weight of the composition. The emulsifiers and co-emulsifiers used in the composition can be chosen from among those conventionally used in the field under consideration. For example, they can be used in a proportion going from 0.3 to 30% by weight with relation to the total weight of the composition.

### X. In Vitro Uses

In addition to treatment methods and other in vivo uses, the penetrating peptide compositions disclosed herein can also be used in the context of in vitro experimentation. For example, the penetrating peptides disclosed herein can be used to deliver any of a wide variety of active agents as discussed herein, as well as potential active agents, into viable cells in vitro to determine the potential therapeutic effect, toxicity, etc. of the active agent or potential active agent. For this reason, the penetrating peptides and penetrating peptide compositions of the present disclosure can be useful in the context of drug testing and/or screening.

In some embodiments, penetrating peptide compositions as described herein can be used in in vitro gene silencing experiments, e.g., by introducing a penetrating peptide-interfering RNA conjugate directed to a gene target and monitoring the effect on gene expression.

Additional in vitro uses can include the use of penetrating peptides as disclosed herein conjugated or associated with one or more labeling agents (e.g., fluorescent agents or radioactive labels) or one or more labeling agent carriers in order to label viable cells in vitro.

### EXAMPLES

The following examples are put forth so as to provide those of ordinary skill in the art with a complete disclosure and description of how to make and use the presently disclosed subject matter, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### Example 1

### Conjugation of SPACE Peptides to Cyclosporin A

Cyclosporin A (CsA) was conjugated to SPACE Peptides using the generalized scheme depicted in FIG. 1. In Step I, CsA was epoxidized by mixing CsA (50 mg), m-Chloroperoxybenzoic acid (8 mg), and anhydrous Na₂CO₃ (10 mg) in 5 mL of methylene chloride. The reaction mixture was stirred overnight at room temperature (RT). The reaction mixture was thereafter washed with 20% sodium bisulfite and 10% sodium carbonate. The organic layer was dried over anhydrous sodium carbonate. The solvent was removed under vacuum producing a crystalline product.

In Step II, a SPACE Peptide having the amino acid sequence set forth in SEQ ID NO: 13 was conjugated at its N-terminus to the epoxide of CsA obtained from Step I above. The epoxide of CsA was dissolved in 5 ml of ethanol at RT overnight. 50 mg of SPACE Peptide was added and the mixture was incubated overnight at RT to form a SPACE Peptide/CsA conjugate.

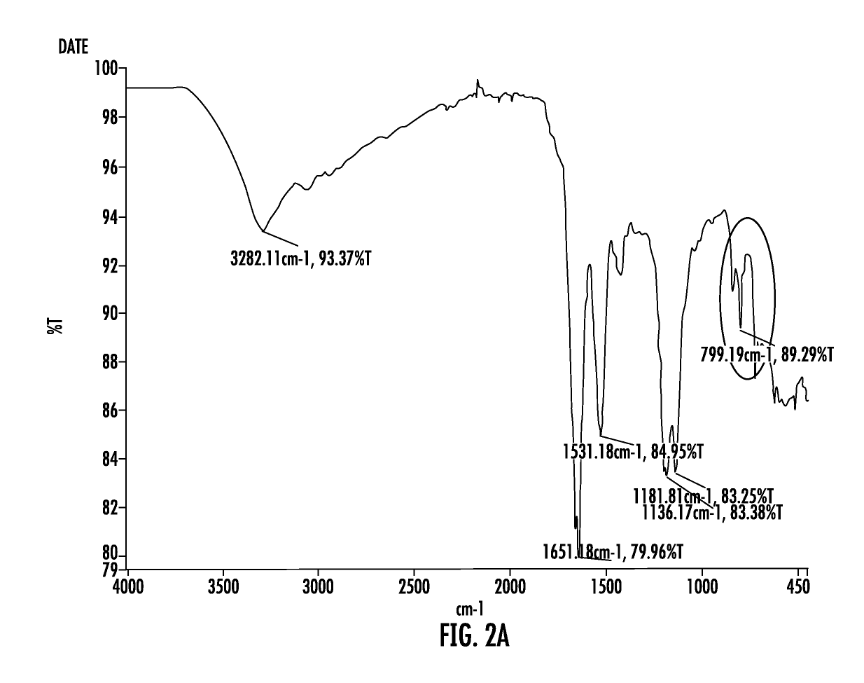

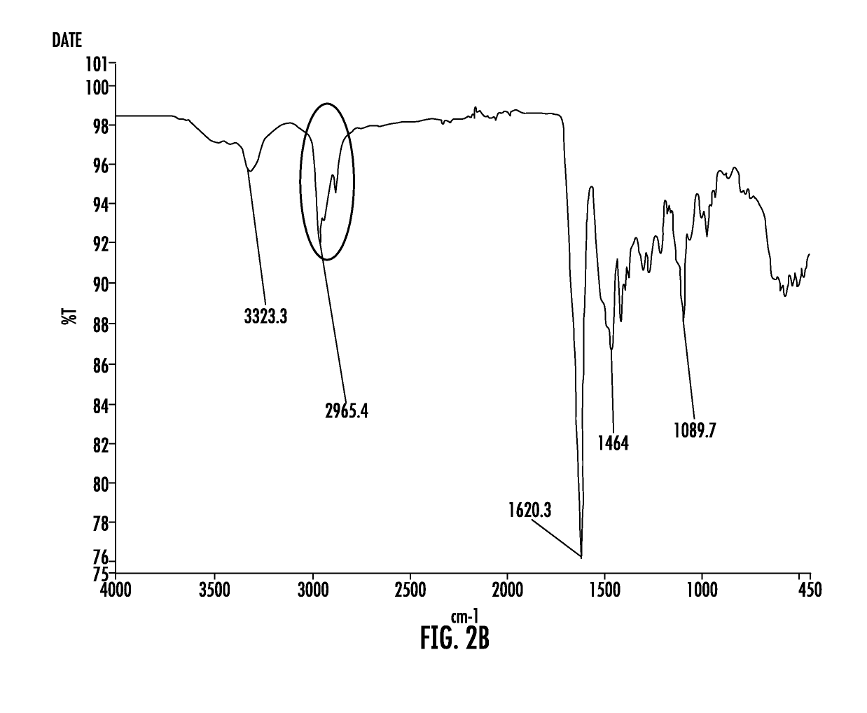

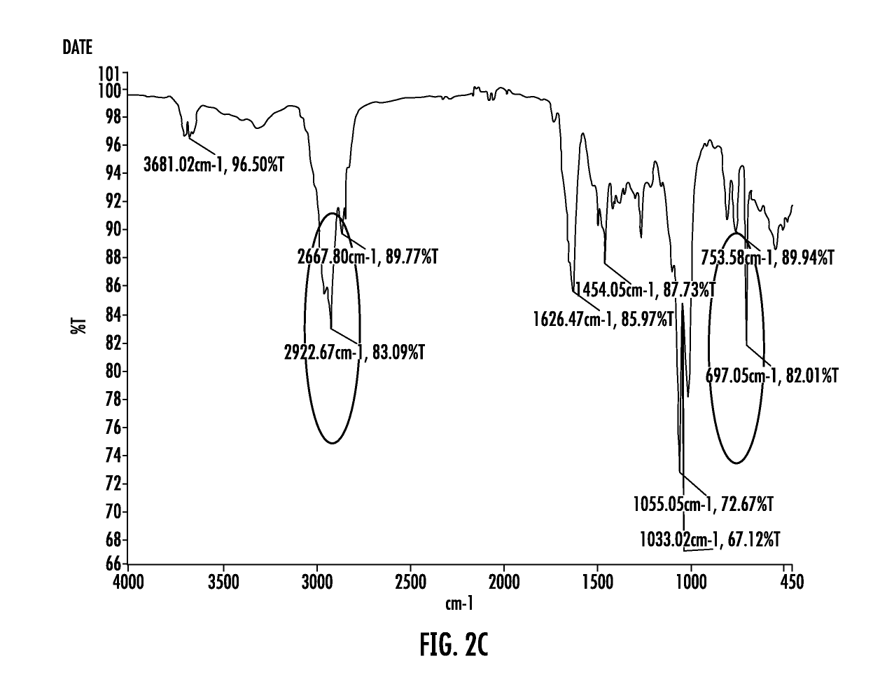

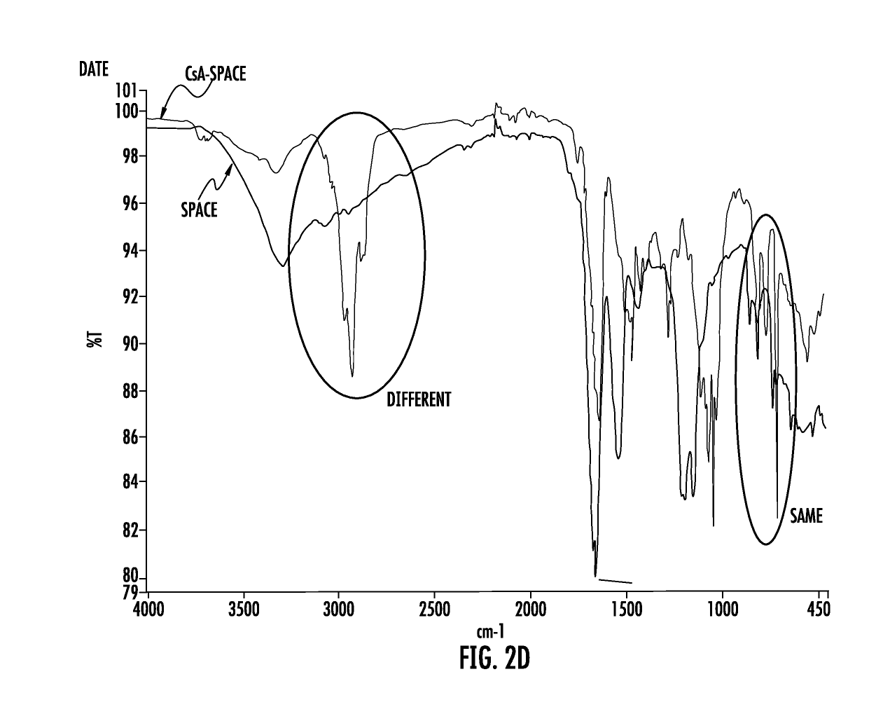

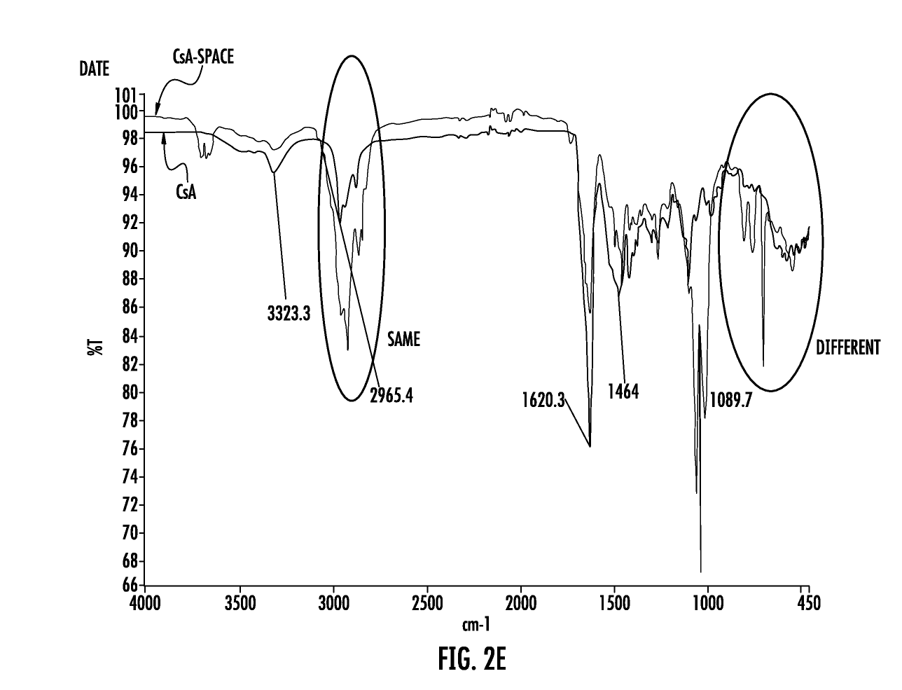

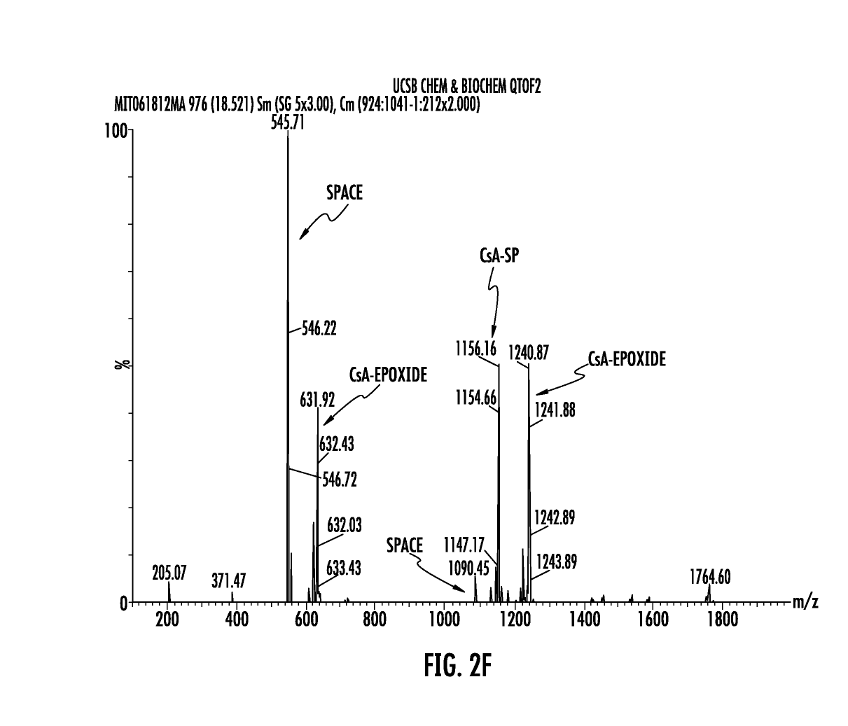

FIGS. 2A-2C show FTIR spectra of SPACE Peptide powder (FIG. 2A), CsA powder (FIG. 2B), and a SPACE Peptide-CsA conjugate (FIG. 2C). The characteristic spectra are used to determine the presence of both components in the conjugate as determined by the presence of characteristic features in the spectra. Comparison of the spectrum of the SPACE Peptide-CsA conjugate with either SPACE alone (FIG. 2D) or CsA alone (FIG. 2E) confirmed the presence of both components. Since the SPACE Peptide was highly water soluble and cyclosporin was highly water insoluble, the simultaneous presence of both components indicated their close association.

Mass spectrometry was also performed to confirm these conclusions. FIG. 2F directly confirmed the presence of cyclosporin-SPACE conjugate via a mass spectrometry trace of reactants (SPACE Peptide (SPACE)), an epoxide of cyclosporin A (CsA-Epoxide)), and a product (SPACE Peptide-conjugated cyclosporin A (CsA-SP)).

### Example 2

### In Vitro Skin Penetration Studies

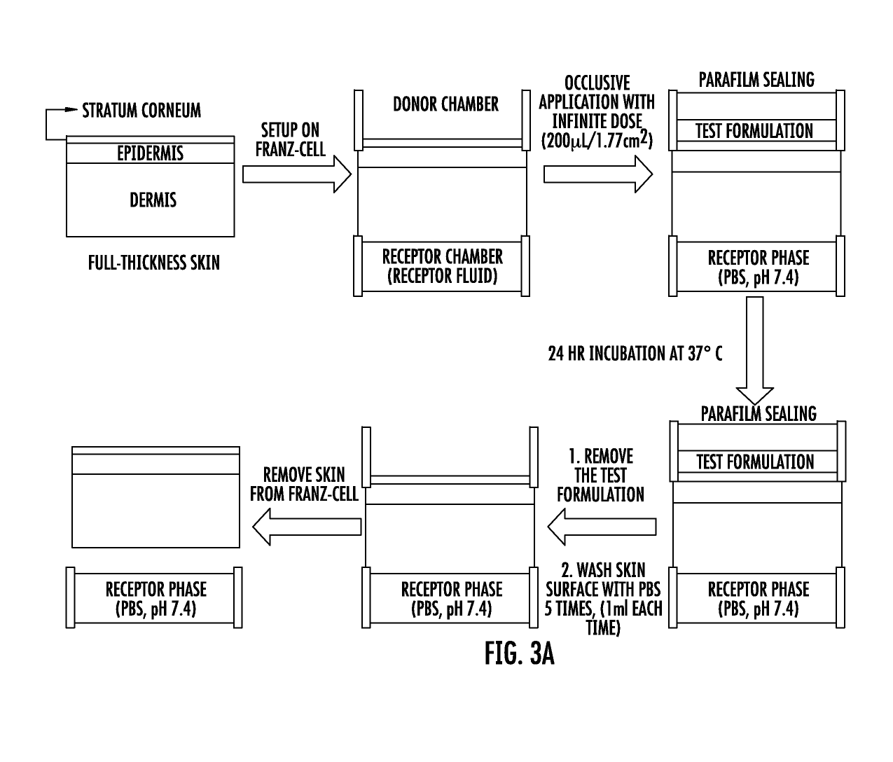

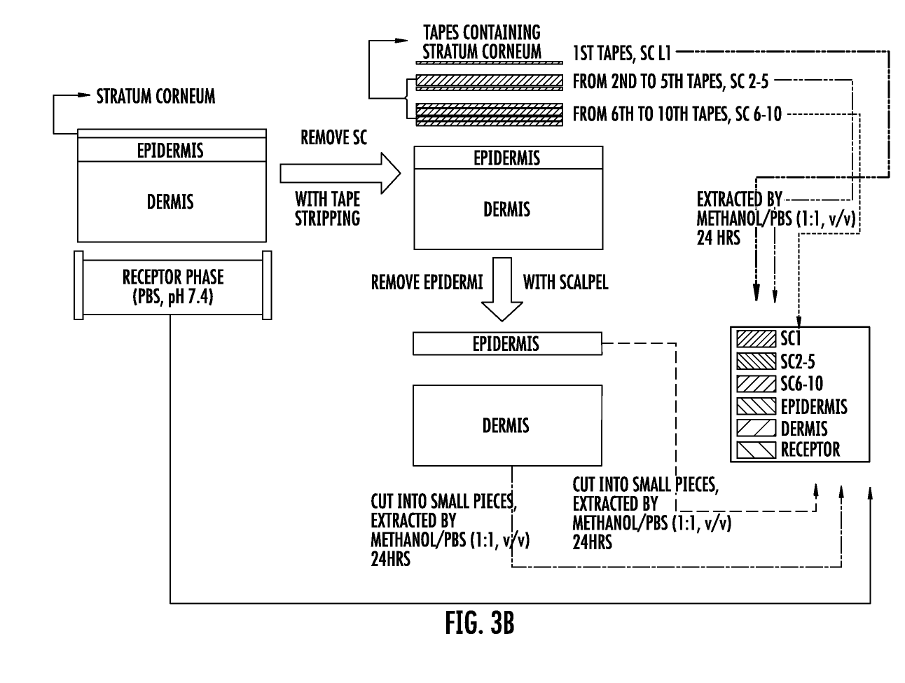

In vitro studies were employed to test the ability of SPACE Peptides and conjugates thereof to penetrate the skin. A generalized scheme for performing these in vitro studies is depicted in FIG. 3.

Full thickness pig skin (Lampire Biological Laboratories, Pipersville, Pa.) was used. The skin was stored at −80° C. and defrosted immediately prior to use. Briefly, the skin was allowed to thaw with the stratum corneum (SC) side up left open to the atmosphere. Skin disks of 36 mm were punched out. The subcutaneous fatty tissue was carefully removed from the dermis, and the hair shaft was clipped to no more than 4 mm. The skin pieces were cleaned with PBS (pH 7.4). Subsequently, the integrity of the skin disks were checked with a skin conductivity measurement to ensure that samples were free from any surface irregularity such as tiny holes or crevices in the portion that was used for skin penetration and deposition studies.

In vitro skin penetration and deposition experiments of different liposomal systems containing a SPACE Peptide labeled with fluorescein isothiocyanate (FITC-SPACE), CsA, or fluorescent hyaluronidase (Fluorescein-HA) were run in Franz diffusion cells occlusively and maintained at 37±1° C. throughout experiments using an oven. The effective penetration area and receptor cell volume were 1.77 cm² and 12.0 ml, respectively. The acceptor compartment was filled with PBS pH 7.4 as the receptor medium. Each test formulation was investigated in triplicate. Skin disks were mounted with the SC side up and the donor compartment left dry and open to atmosphere for 0.5 hour before test formulation application. Caution was taken to remove all air bubbles between the underside of the skin (dermis) and the acceptor solution. The skin was stretched in all directions to avoid the presence of furrows.

100-200 μL of the test formulation was applied to skin surface using a pipette. The experiments were carried out under occlusion with light protection. The incubation time of the skin with each test formulation was 24 hours. At the end of an experiment, a sample of 1-3 mL was withdrawn from the receptor phase for concentration measurement by fluorescence assay using a micro-plate reader (SAFIRE, XFLUOR4, V4.50, Tecan Group Ltd, NY, United States of America) for fluorescence. In the case of CsA experiments, a sample of 3 ml was withdrawn from the receptor phase for ³H-CsA measurement by a liquid scintillation counter (TRI-GARB 2100TR, Packard Instrument Company, Downers Grove, Ill.). The formulations were removed from the skin by washing five times with PBS (pH 7.4). After cleaning, the skin was transferred onto a device for tape-stripping the SC.

Extraction of Drug from Skin Layers:

The stratum corneum was removed by striping with an adhesive tape (SCOTCH® Transparent Tape, 3M Corporate, St. Paul, Minn.). In order to avoid any furrows, which could be a reason for false results of the stripping procedure, the skin was stretched and mounted on cork discs as mentioned previously. The skin was covered with a TEFLON® mask with a central hole of 15 mm in diameter. Adhesive tape was put onto the skin and a weight of 2 kg was placed on the tape for 10 seconds. The tape was rapidly removed with forceps and transferred into a glass vial of suitable size. Ten stripping procedures were performed consecutively. For analytical reasons, the stripped tapes were collected in glass vials according the following scheme: vial 1=strip 1^{st}, vial 2=strip 2^{nd}-5^{th}, and vial 3=strip 6^{th}-10^{th}. After tape-stripping, the epidermis sheet was separated from the dermis with a sterile surgical scalpel and cut into small pieces and collected into a glass vial. Dermis was thereafter cut into small pieces and transferred into a glass vial.

For extraction of drug from the separated skin layers, in the case of FITC-SPACE and Fluorescein-HA, 4 ml of methanol and PBS pH 7.4 (1:1, v/v) mixture was added to each glass vial. The vials were shaken at 200 rpm on an orbital shaker overnight at room temperature. The dispersions were centrifuged (10 min, 10000 rpm) to subside skin tissue pieces at the bottom. The supernatant was withdrawn, diluted if the concentration was found outside the range of detection, and analyzed by fluorescence measurement. In the CsA experiments, 5 ml of SOLVABLE™, an aqueous tissue solubilizer (PerkinElmer, Inc., Walthan, Mass.), was added to each vial. The vials were kept at 60° C. overnight and cooled down at room temperature. 5 ml of liquid scintillation cocktail (ULTIMA GOLD™, PerkinElmer, Inc., Walthan, Mass.) was added and ³H-CsA was analyzed by the liquid scintillation counter (TRI-GARB 2100TR, Packard Instrument Company, Downers Grove, Ill.).

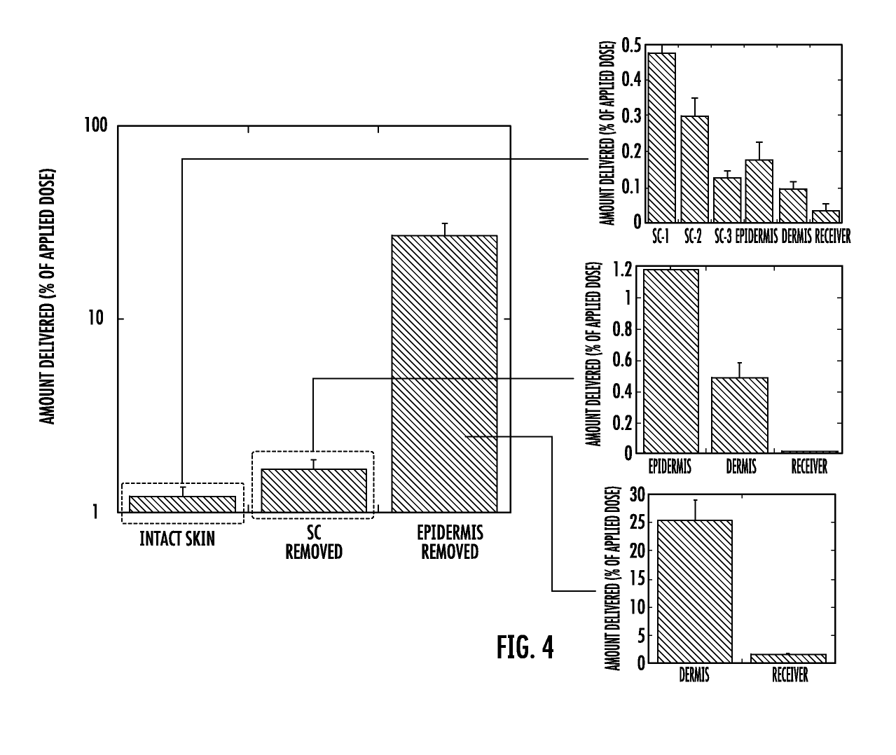

FIG. 4 summarizes the results of the tests of the transport barrier in the skin for a fluorescently-labeled SPACE Peptide (FITC-SPACE; SPACE Peptide employed was SEQ ID NO: 13). Labeling with FITC was done during peptide synthesis. The results of three representative tests are presented; (i) placement of SPACE Peptide on intact porcine skin; (ii) placement of SPACE Peptide on skin after the SC was removed by tape stripping, thus exposing the epidermis of the skin; and (iii) placement of SPACE Peptide on skin after SC and epidermis were removed, thus exposing the dermis of the skin. The first case represented normal skin. The second case represented a disease where the SC was compromised, and the third case represented an extreme case where the epidermis was missing, for example, open wounds.

A comparison of penetration in these cases indicated that the SC provided the primary barrier to transport. More importantly, these data confirmed that regardless of the extent of skin's barrier, SPACE Peptide showed a greater than 100-fold higher concentration of FITXC-SPACE Peptide in the dermis as compared to that in the receiver compartment.

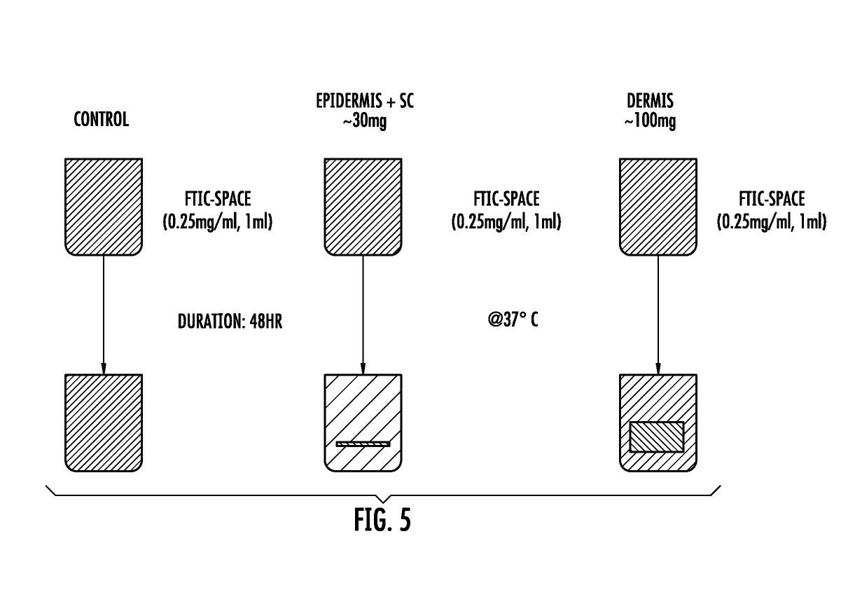

The localization effect of SPACE Peptide can be seen even clearly from FIG. 5, which shows a partitioning effect of the peptide in the skin. In these experiments, epidermis and dermis from porcine skin were isolated and placed in separate vials in an aqueous solution of SPACE Peptide of SEQ ID NO: 13. The amount of SPACE Peptide that partitioned into each skin layer was measured after 24 hours. The ratio of concentrations in skin layers and surrounding PBS was used to determine the partition coefficient. The data show that SPACE Peptide exhibited a partition coefficient of 9.8 in the epidermis and 4.3 in the dermis.

### Example 3

### Synthesis of Exemplary SPACE Peptide-Conjugated Lipids

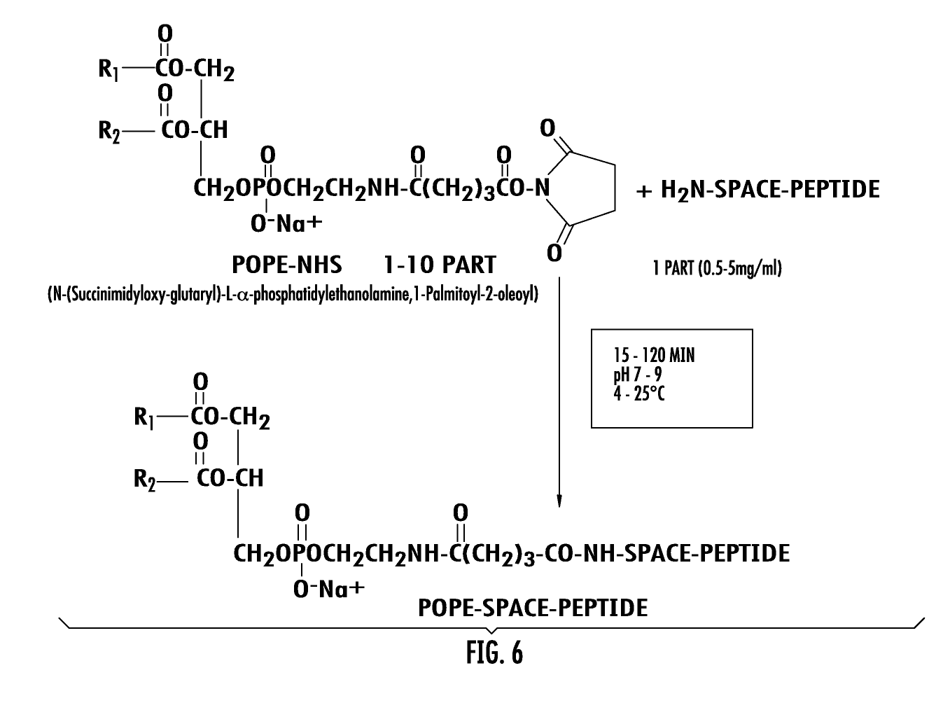

SPACE Peptide-conjugated lipids were synthesized using the basic procedure described herein below and depicted in FIG. 6.

### Materials.

Phospholipon 90G (American Lecithin Company, Oxford, Conn.)
POPE-NHS (NOF America Corporation, White Plains, N.Y.)
SPACE Peptide (ACTGSTQHQCG (SEQ ID NO: 13, with a disulfide bridge between amino acids 2-10) (Ambiopharm, North Augusta, S.C.)
FITC-SPACE Peptide (FITC-Ahx-ACTGSTQHQCG (SEQ ID NO: 13, with a disulfide bridge between amino acids 2-10) (Ambiopharm, North Augusta, S.C.)
Fluorescein Hyaluronic acid (molecular weight 200-325 kDa, Creative PEGWorks, Winston Salem, N.C.)
Cyclosporin A (Abcam, Cambridge, Mass.)
³H-Cyclosporin A (American Radiolabeled Chemicals, Inc., St. Louis, Mo.)

### Methods:

Conjugation of SPACE with POPE-NHS:

0.5 ml of SPACE Peptide (4 mg/mL in PBS, pH 8.0) was incubated with 0.5 mL of POPE-NHS (4 mg/ml in ethanol) at room temperature for 2 hours (hereinafter the “POPE-NHS reaction solution”).

Confirmation of the Conjugation of SPACE Peptide with Liposomes:

The conjugation of SPACE Peptide with POPE-NHS was confirmed by the 2,4,6-Trinitrobenzene sulfonic acid (TNBS) method of Chang et al. (2009) 4 PLoS ONE e4171. The TNBS method is based on the ability of TNBS to interact with primary amino groups of peptides to generate a highly chromogenic product which can be readily measured at 335 nm. If the SPACE Peptide was successfully conjugated to POPE-NHS, there would be no primary amino group remaining available to TNBS, the chromogenic product would not be generated, and no signal would be detected at 335 nm.

Briefly, 50 μL of SPACE Peptide and POPE-NHS reaction solution (100 μg of SPACE Peptide involved in the reaction system or 50 μL of free SPACE Peptide (0-200 μg of SPACE Peptide) was diluted with 450 μL of 0.1 M sodium bicarbonate solution (pH 8.5). 250 μL of working solution of TNBS (1% in 0.1 M sodium bicarbonate solution, pH 8.5) was added and incubated at 37° C. for 2 hrs. Afterwards, 250 μL of 10% SDS and 125 μL of 1 M HCl was added to stop the reaction. Finally, absorbance was measured at 335 nm.

### Example 4

### Preparation of SPACE-Peptide Ethosomes

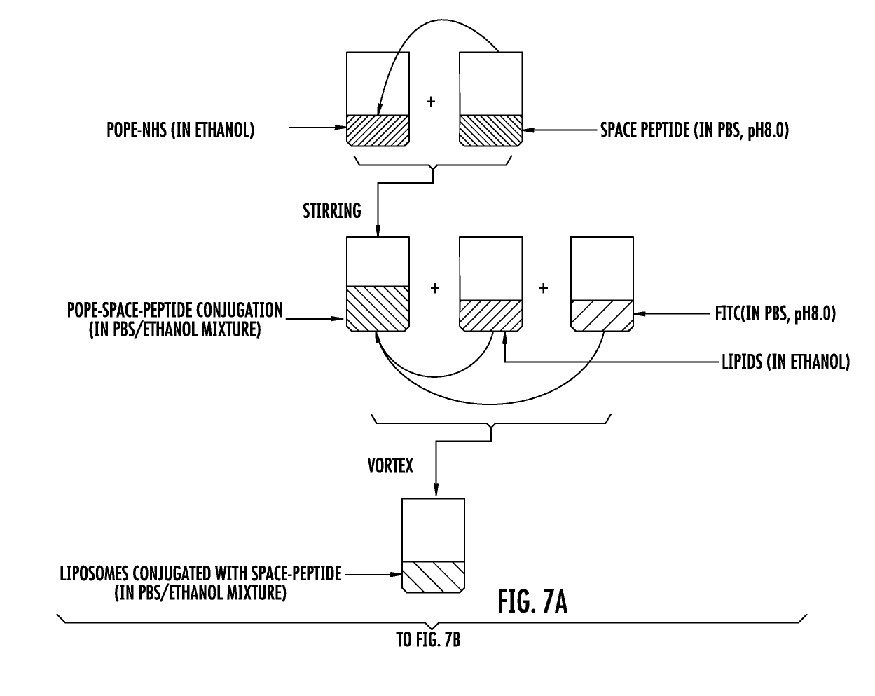

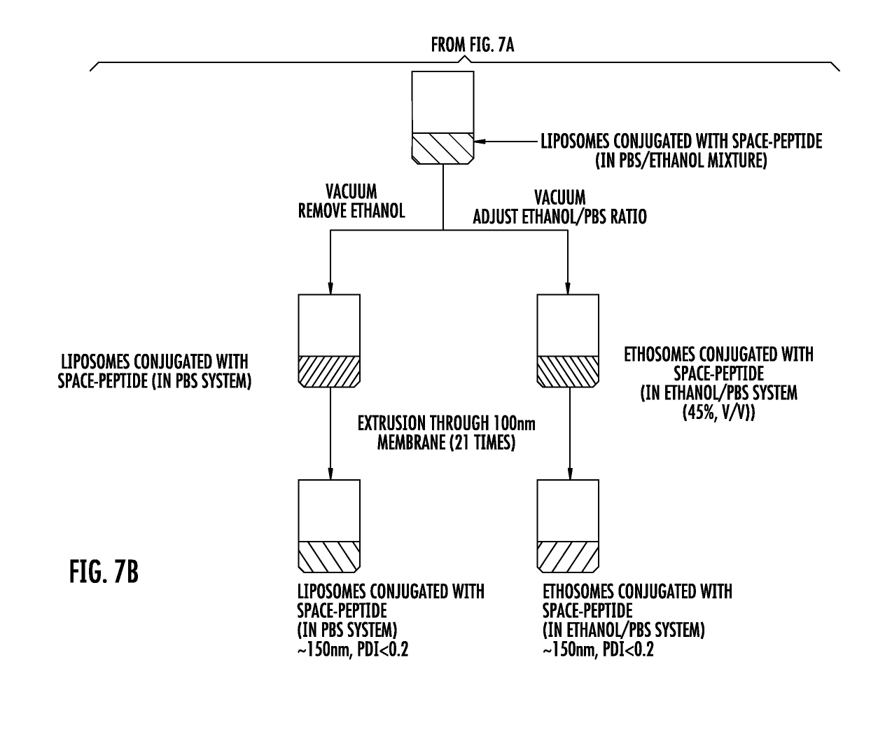

SPACE Peptide-conjugated lipids were used to prepare SPACE-Peptide-displaying ethosomes. A general procedure for preparing SPACE-Peptide-displaying ethosomes is presented in FIGS. 7A and 7B.

For 1 ml of ethosomes, 40 mg of Phospholipon 90G was dissolved in 2 ml ethanol and added into the SPACE-POPE conjugation solution obtained as per the EXAMPLE 3. The solvent (both ethanol and water) system was removed using a rotary evaporator at room temperature. To produce fluorescent SPACE-Peptide-displaying ethosomes, 1 ml of 45% ethanol/water (v/v) containing 1 mg of FITC-SPACE and 50 mg of free SPACE Peptide was used to hydrate the lipid film. To produce fluorescent SPACE-Peptide-displaying ethosomes carrying hyaluronic acid, 1 ml of ethanol/acetic acid buffer (pH 4.0; 45%, v/v) mixture or 1 ml of ethanol/water (45%, v/v) mixture containing 1 mg of fluorescein-labeled hyaluronic acid (fHA) and 50 mg of free SPACE Peptide was used to hydrate the lipid film. The obtained ethosomal solutions were extruded 21 times through a 100 nm polycarbonate membrane using a mini-extruder.

### Example 5

### Delivery of Fluorescent Labels to Skin Layers Using SPACE Peptide Conjugates

The concentrations of FITC-SPACE and fHA were determined by fluorescence spectroscopy. Fluorescence detection was performed at an excitation of 485 nm and an emission of 520 nm for both. The method was validated for linearity, accuracy, and precision. The linear range during the measurements for FITC-SPACE and fHA was from 0.005 μg/mL to 0.5 μg/mL (r²=0.9999) and from 0.01 μg/mL to 10 μg/mL (r²=0.9999), respectively.

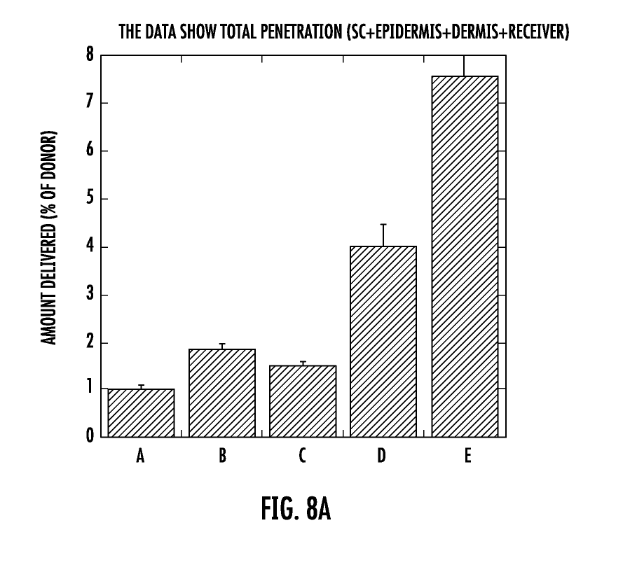

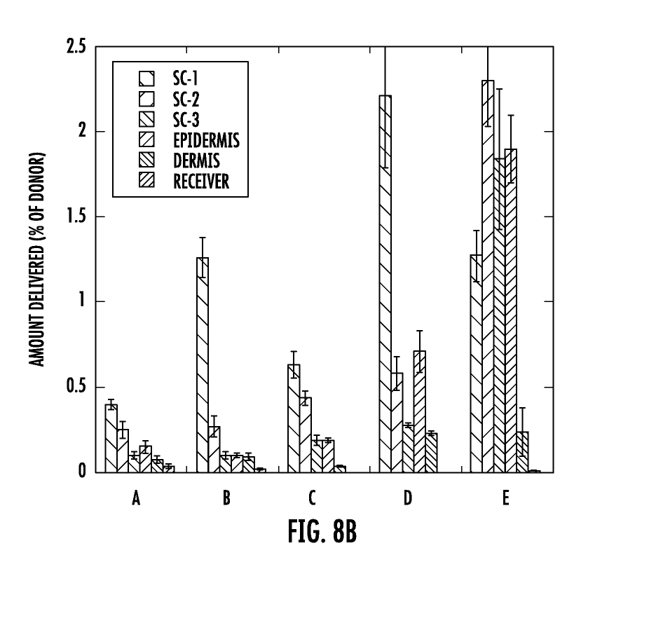

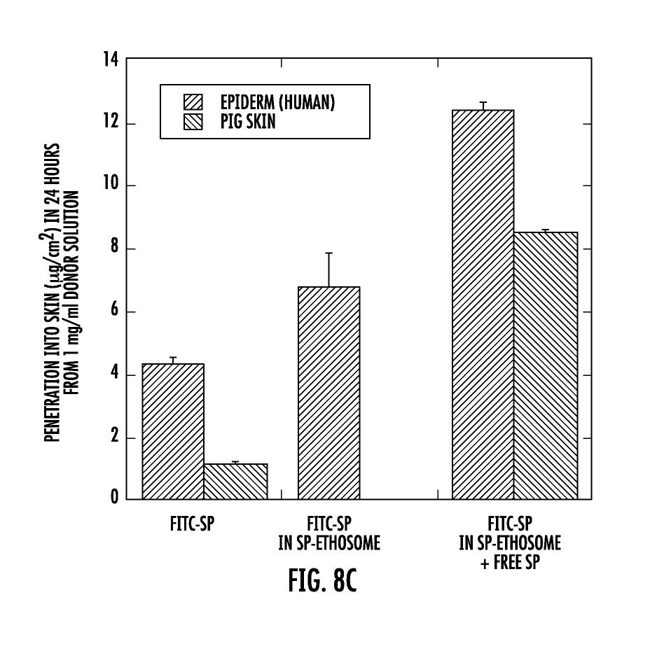

Formulations comprising fluorescent SPACE Peptide were prepared and tested using the methods presented herein above, the results of which are summarized in FIG. 8.

FIG. 8A is a bar graph showing total penetration of various SPACE Peptide-containing formulations in SC+Epidermis+Dermis+Receiver. In the bar graph, column A presents data for FITC-SPACE at 1 mg/ml; column B presents data for FITC-SPACE at 1 mg/ml+free SPACE Peptide at 10 mg/ml; column C presents data for SPACE-lipids containing FITC; column D presents data for SPACE-ethosomes containing FITC; and column E presents data for SPACE-ethosomes containing FITC-labeled SPACE Peptide+25 mg/ml free SPACE Peptide.

Taken together, the data presented in FIG. 8A demonstrated that SPACE-ethosomes, in the presence of free SPACE Peptide of 25 mg/ml, delivered about 7.5% of the applied dose to the skin in 24 hours. Of note is that the experiments summarized in FIG. 8A were performed under infinite dosing conditions.

FIG. 8B shows that about 2% of the applied does was deposited in epidermis. Additional experiments performed using EPIDERM™ tissue confirmed penetration of SPACE Peptide across the skin. For this purpose, the SPACE Peptide formulations were prepared using the methods described herein above. EPIDERM™ tissues were obtained from MatTek Corporation (Ashland, Mass.). The EPIDERM™ tissue possessed cornified stratum corneum that was backed by viable epidermis. Thus, this tissue had a combination of barrier properties and living cells. SPACE Peptide showed excellent penetration from SPACE-ethosome formulations across EPIDERM™ tissue. A 100 microliter formulation applied on EPIDERM™ tissue for 24 hours was determined to deliver 25% of SPACE Peptide across EPIDERM™ tissue. The data presented in FIG. 8C showed that a FITC-labeled SPACE Peptide penetrated into the EPIDERM™ tissue, encapsulation of the FITC-labeled SPACE Peptide into a SPACE Peptide-conjugated ethosome further enhanced penetration in to the EPIDERM™ tissue, and the addition of a free SPACE Peptide into the formulation further enhanced skin penetration both in the EPIDERM™ tissue and in the pig skin model.

Summarizing FIG. 8B, it was noted that:
The highest penetration was found in the superficial SC layer;
Free SPACE Peptide enhanced penetration of FITC-SPACE Peptide formulations;
Liposomes enhanced penetration into superficial layers but were less effective for deeper layers;
Ethosomes enhanced penetration into SC and Epidermis;
SPACE-ethosomes carrying FITC-SPACE Peptide exhibited the highest delivery into all skin layers; and
Penetration into the Epidermis was as high as that in SC.

FIG. 8C is a bar graph showing delivery of a fluorescent label to EPIDERM™ (left column of each pair) and a pig skin model (right column of each pair) using different formulations the contain SPACE Peptides. A 100 μl sample of various formulations was placed on a 2 cm² skin sample and penetration was tested 24 hours after application. As shown in FIG. 8C, a FITC-labeled SPACE Peptide penetrated into EPIDERM™, encapsulation of the FITC-labeled SPACE Peptide into a SPACE Peptide-conjugated ethosome further enhanced penetration in to EPIDERM™, and the addition of a free SPACE Peptide into the formulation further enhanced skin penetration both in EPIDERM™ and in the pig skin model.

### Example 6

### Delivery of Cyclosporin A to Skin Layers Using SPACE-Peptide Conjugates

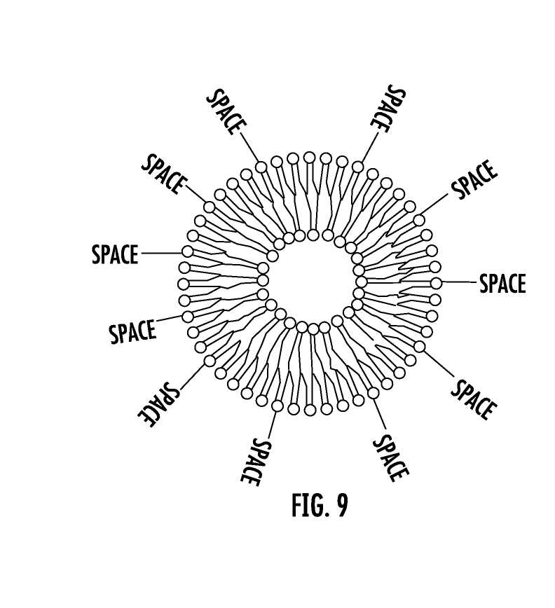

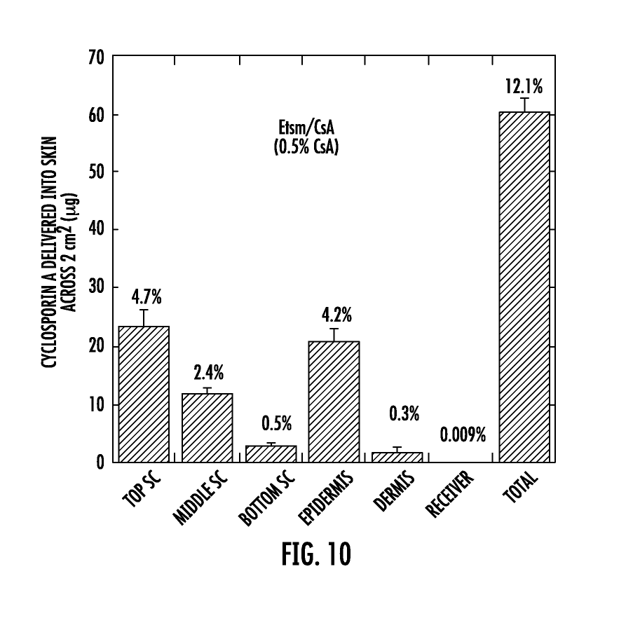

SPACE ethosomes were used to encapsulate cyclosporin. SPACE ethosomes were prepared using the procedure outlined in EXAMPLE 3 and its penetration into skin was measured using the procedure outlined in EXAMPLE 2. The resultant ethosomes possessed a diameter of about 150 nm and possessed a zeta potential of −50 mV (see FIG. 9). SPACE-ethosomes delivered substantial amounts of cyclosporin into skin (see FIG. 10). Specifically, about 12.1% of topically applied cyclosporin was determined to penetrate into skin after 24 hours when 100 μL of formulation was applied to 2 cm². A significant quantity (4.2%) penetrated into epidermis. Further, cyclosporin A exhibited substantial localization in the skin. The amount of cyclosporin A in the skin was 1284-fold higher than that in the receiver compartment.

Of note is that based on the current literature, the therapeutic concentration of CsA delivered intralesionally is typically in the range of 2-35 μg, which is generally reached only after a 12-injection course over 4 weeks. The data presented in FIG. 10 demonstrated that single doses of a 0.5% CsA-containing SPACE ethosome could deliver between 0.3% (dermis) and 4.7% (top SC) of the CsA. 0.5% CsA corresponds to 500 μg/100 μl, meaning that between 1.5 μg and 23.5 μg of CsA were delivered to various skin compartments in 24 hours using CsA-containing SPACE ethosomes.

### Example 7

### Delivery of Hyaluronic Acid to Skin Layers Using SPACE-Peptide Conjugates

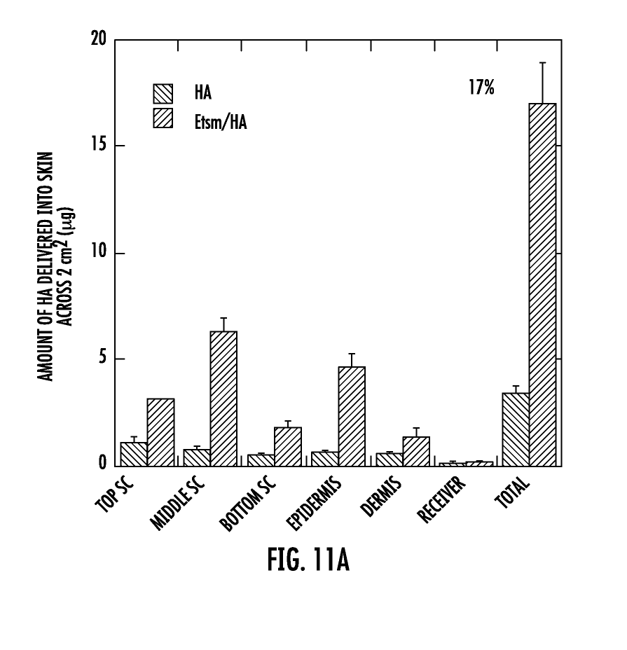

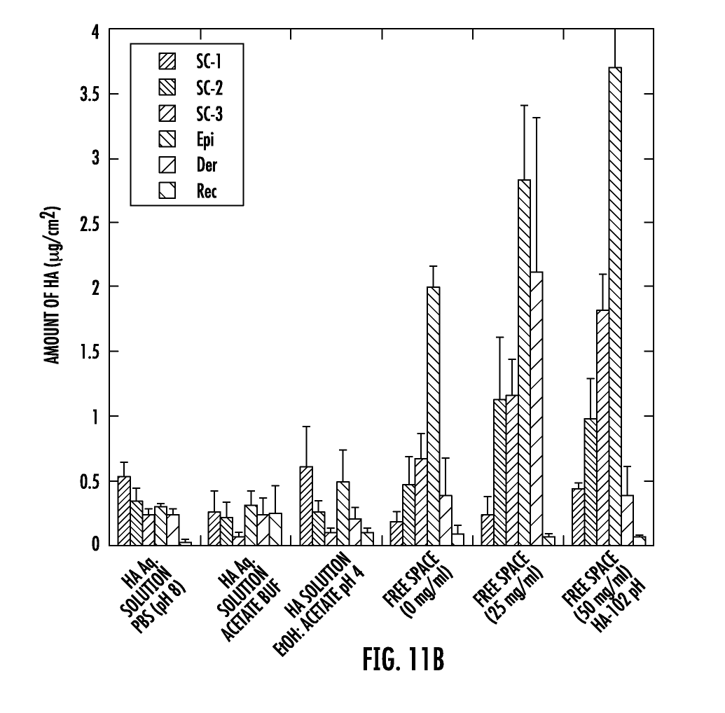

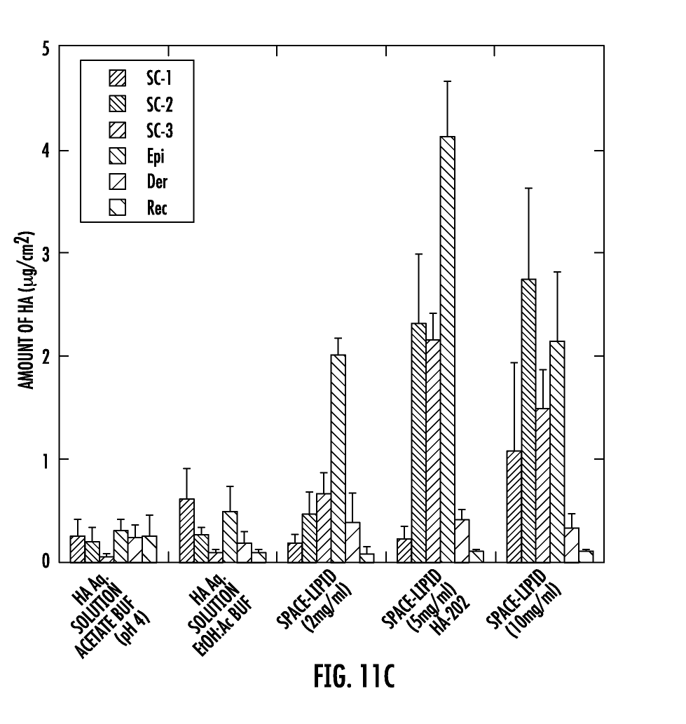

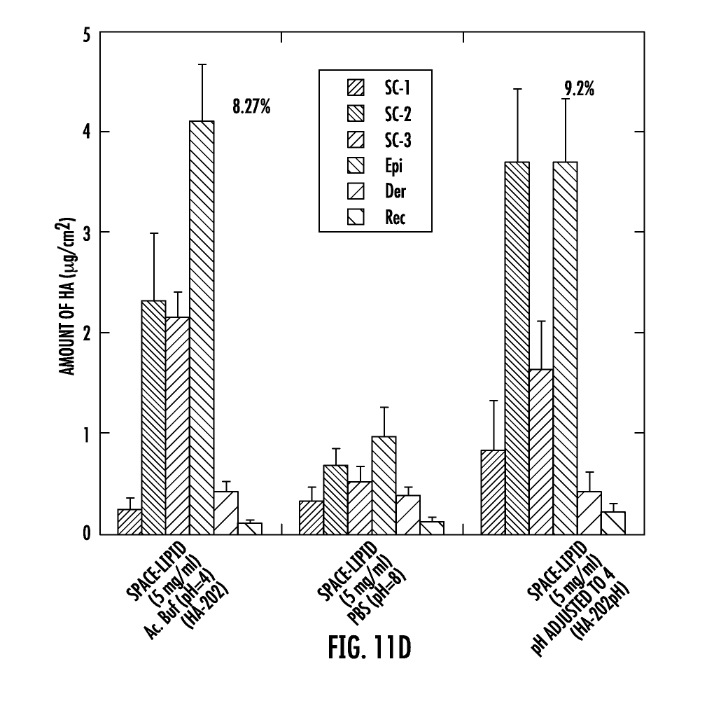

SPACE ethosomes were used to encapsulate HA. SPACE ethosomes were prepared using the procedure outlined in EXAMPLE 3 and its penetration into skin was measured using the procedure outlined in EXAMPLE 2. As shown in FIG. 11A, SPACE-ethosomes delivered substantial amounts of HA into skin. Specifically, about 17% of topically-applied HA was determined to penetrate into skin when 100 μL of formulation was applied to 2 cm² for 24 hours. A significant quantity also penetrated into epidermis: an 8-fold enhancement of epidermal accumulation was found compared to an aqueous solution of HA. Further, cyclosporin A exhibited substantial localization in the skin. The amount of HA in the skin was significantly higher than that in the receiver compartment.

The effect of SPACE-Peptide concentration in the formulation on HA delivery was explored. These formulations were prepared using methods described in EXAMPLE 3 and tested using methods described in EXAMPLE 2. These formulations were prepared in the acetate buffer at pH 4. As shown in FIG. 11A, significant penetration of HA into skin was found. In particular, HA was found to penetrate into epidermis and dermis of the skin. Penetration of HA from the ethosomal formulation was significantly higher than that from a control (aqueous solution of HA at the same concentration). Ethosomal HA led to about 8-fold higher penetration into epidermis compared to control.

FIG. 11B shows the effect of free SPACE concentration on HA delivery from SPACE-ethosome formulations. For these experiments, the concentration of SPACE-lipid in all formulations was fixed at 2 mg/ml and all SPACE formulations were prepared using acetate buffer (pH 4) and ethanol. Compared to controls, which led to HA delivery of less than 0.5 microgram per sq. cm in the epidermis, SPACE-ethosome formulations delivered significantly higher amounts of HA. The delivery increased with increasing concentrations of free SPACE Peptide. While SPACE-ethosomes without free SPACE delivered about 2 micrograms of HA per sq. cm, increasing the free SPACE concentration to 50 mg/ml increased the delivered amount to more than 3.5 micrograms per sq. cm.

The effect of SPACE Peptide concentration in the lipid-conjugated form of HA delivery was also assessed (see FIG. 11C). The free SPACE Peptide concentration in the formulations was 0 mg/ml, and all SPACE formulations were prepared using acetate buffer (pH 4) and ethanol. Of the conditions tested, a SPACE-lipid concentration of 5 mg/ml yielded the best delivery, with about 4 micrograms of the applied dose entered the skin per sq. cm.

In another embodiment, the pH of the formulation was adjusted to 4 by addition of hydrochloric acid (referred to as “HA-202pH” in FIG. 11D). Excellent penetration of HA from this formulation into epidermis was seen (see FIG. 11D). While not wishing to be bound by any particular theory of operation, it appeared that pH played a role in the performance of HA from the formulations since the formulation made at pH 8 delivered less HA than an otherwise identical formulation at pH 4.

### Example 8

### In Vitro Delivery of siRNAs to Skin Using SPACE-Ethosomes

SPACE ethosomes were also tested for their ability to deliver siRNAs to skin using the following general procedure.

### Materials:

DOTAP (Avanti Polar Lipids, Inc., Alabaster, Ala.)
POPE-NHS(NOF America Corporation, White Plains, N.Y.)
SPACE Peptide (ACTGSTQHQCG (SEQ ID NO: 13), with a disulfide bridge between amino acids 2-10) (Ambiopharm, North Augusta, S.C.)
FAM Labeled GAPDH-siRNA (5′-FAM-GAC GUA AAC GGC CAC AAG UUC-3′ (SEQ ID NO: 19), Ambion, Life Technologies, Grand Island, N.Y.)
Modified FAM-GAPDH-siRNA (5′-FAM-GAC GUA AAC GGC CAC AAG UUC N6-3′ (SEQ ID NO: 19), Dharmacon, Thermo Fisher Scientific, Inc. Waltham, Mass.)

### Method:

Conjugation of SPACE with POPE-NHS:

0.5 ml of SPACE Peptide (4 mg/mL in PBS, pH 8.0) was incubated with 0.5 mL of POPE-NHS (4 mg/mL in ethanol) at room temperature for 2 hrs.

Confirmation of the Conjugation of SPACE Peptide with Liposomes:

The conjugation of SPACE Peptide with POPE-NHS was confirmed by the TNBS method as described herein above. Briefly, 50 μL of SPACE Peptide and POPE-NHS reaction solution (containing 100 μg of SPACE Peptide) or 50 μL of free SPACE Peptide (containing 0-200 μg of SPACE Peptide) was diluted with 450 μL of 0.1 M sodium bicarbonate solution, pH 8.5). 250 μL of working solution of TNBS (1% in 0.1 M sodium bicarbonate solution, pH 8.5) was added into above sample solution and incubated at 37° C. for 2 hrs. Afterwards, 250 μL of 10% SDS and 125 μL of 1 M HCl were added to stop the reaction. Finally, the absorbances from the conjugation reaction group and from a standard samples group were measured at 335 nm.

Conjugation of GAPDH-siRNA and SPACE:

A 10 mM SPACE Peptide solution was incubated with a 10 mM solution of N-(3-dimethylaminopropyl)-N′-ethylcarbodiimide hydrochloride (EDAC, Sigma) and a 9.5 mM solution of N-hydroxysulfosuccinimide sodium salt (NHS, Sigma) in equal parts in MES buffer (pH 5.5) for 15 min. An amine-modified siRNA was then added and mixed overnight to conjugate the peptide to the siRNA.

Preparation of DOTAP-Ethosomes Containing GAPDH-siRNA or Conjugation of GAPDH-SiRNA and SPACE:

For 1 ml of ethosomes, 10 mg of DOTAP and 2 mg of cholesterol was dissolved in 2 ml ethanol and added into SPACE-POPE conjugation solution prepared as described herein above. The solvent (both ethanol and water) system was removed using a rotary evaporator at room temperature. Afterwards, 1 ml of ethanol/acetic acid buffer (45%, v/v; pH 4.0 for whole mixture solution), which contained 25 nmol of GAPDH-siRNA and 50 mg of free SPACE Peptide, or 1 ml of ethanol/MES buffer (45%, v/v; pH 4.0 for whole mixture solution), which contained 25 nmol of GAPDH-siRNA-SPACE conjugation and 50 mg of free SPACE Peptide, was used to hydrate the lipid film. The obtained ethosomal solution was extruded 21 times through a 100 nm polycarbonate membrane using a mini-extruder.

Skin Preparation:

Full thickness pig skin (Lampire Biological Laboratories, Pipersville, Pa.) was used. The skin was stored at −80° C. and defrosted immediately prior to use. Briefly, the skin was allowed to thaw with the stratum corneum side up left open to the atmosphere for at least half hour. Skin disks of 36 mm were punched out. The subcutaneous fatty tissue was carefully removed from the dermis, and the hair shaft was cut off to no more than 4 mm. The skin piece was cleaned with PBS (pH 7.4). The integrity of skin disks was checked with a skin conductivity measurement to ensure that the samples were free from any surface irregularities such as tiny holes or crevices in the portion that was used for skin penetration and deposition studies.

Franz Diffusion Cell Setup:

In vitro skin penetration and deposition experiments of different formulations were run in Franz diffusion cells occlusively and maintained at 37±1° C. throughout the experiments. The effective penetration area and receptor cell volume were 1.77 cm² and 12.0 ml, respectively. The acceptor compartment was filled with PBS buffer pH 7.4 as the receptor medium. Each test formulation was investigated in triplicate. Skin disks were mounted with the SC side up and the donor compartment left dry and open to atmosphere for 0.5 hour before test formulation application. Caution was taken to remove all air bubbles between the underside of the skin (dermis) and the acceptor solution. The skin was stretched in all directions to avoid the presence of furrows. 100 μL of the test formulation was applied to skin surface by a pipette. The experiments were carried out under occlusion with light protection. The incubation time of the skin with different test formulations was 24 hours. At the end of an experiment, a sample of 1 ml was withdrawn from the receptor phase for concentration measurement by fluorescence assay using a micro-plate reader (SAFIRE, XFLUOR4, V4.50, Tecan Group Ltd, NY, US). The formulations were removed from the skin by being washed five times with PBS (pH 7.4). After cleaning, the skin was transferred onto a device for tape-stripping the SC.

Extraction of Drug from Skin Layers:

The stratum corneum was removed by striping with an adhesive tape (SCOTCH® Transparent Tape, 3M Corporate, St. Paul, Minn.). In order to avoid any furrows, which could be a reason for false results of the stripping procedure, the skin was stretched and mounted on cork discs as mentioned herein above. The skin was covered with a TEFLON® mask with a central hole of 15 mm in diameter. Each tape was put onto the skin and a weight of 2 kg was placed on the tape for 10 seconds. Afterwards, the tape was rapidly removed with forceps and transferred into a glass vial of suitable size. Ten stripping procedures were performed consecutively. For analytical reasons, the stripped tapes were collected in glass vials according the following scheme: vial 1=strip 1^{st}, vial 2=strip 2^{nd}-5^{th} and vial 3=strip 6^{th}-10^{th}. After the tape-stripping, the epidermis sheet was separated from the dermis with a surgical sterile scalpel and cut into small pieces and collected into a glass vial. Dermis was also cut into small pieces and transferred into a glass vial. For extraction of drug from the separated skin layers, 4 ml of methanol and PBS pH 7.4 (1:1, v/v) mixture was added to each glass vial. The vials were shaken overnight at 200 rpm on an orbital shaker at room temperature. Afterwards the dispersions were centrifuged (10 min, 10000 rpm) to subside skin tissue pieces at the bottom. The supernatant were withdrawn, diluted if necessary and analyzed by fluorescence measurement.

Fluorescent Assay of FAM-GAPDH-siRNA and FAM-GAPDH-siRNA-SPACE-Peptide Conjugation:

The concentrations of FAM-GAPDH-siRNA and FAM-GAPDH-siRNA-SPACE-Peptide conjugates were determined by fluorescence spectroscopy. Fluorescence detection was performed at an excitation of 495 nm and an emission of 525 nm for both conjugates. The linear ranges during the measurements for FAM-GAPDH-siRNA and FAM-GAPDH-siRNA-SPACE-Peptide conjugation were from 0.25 pmol/mL to 25 pmol/mL (r²=0.9999) and from 0.25 pmol/mL to 25 pmol/mL (r²=0.9999), respectively.

### Example 9

### In Vitro Delivery of siRNAs to Skin Using SPACE-Ethosomes

DOTAP-Ethosomes conjugated with SPACE (2 mg/ml) containing FAM-GAPDH-siRNA (25 nmol/ml) or FAM-GAPDH-siRNA-SPACE (25 nmol/ml)

### Materials:

DOTAP (Avanti Polar Lipids, Inc., Alabaster, Ala.)
POPE-NHS (NOF America Corporation, White Plains, N.Y.)
SPACE Peptide (ACTGSTQHQCG (SEQ ID NO: 13), with a disulfide bridge between amino acids 2-10)) (Ambiopharm, North Augusta, S.C.)
FAM-GAPDH-siRNA (5′-FAM-GAC GUA AAC GGC CAC AAG UUC-3′ (SEQ ID NO: 19), Ambion, Life Technologies, Grand Island, N.Y.) vModified FAM-GAPDH-siRNA (5′-FAM-GAC GUA AAC GGC CAC AAG UUC N6-3′ (SEQ ID NO: 19), Dharmacon, Thermo Fisher Scientific, Inc. Waltham, Mass.)

### Methods:

a. Conjugation of SPACE with POPE-NHS:

0.5 ml of SPACE Peptide (10 mg/ml in PBS, pH 8.0 (0.1 M)) was incubated with 0.5 ml of POPE-NHS (10 mg/ml in Ethanol) at room temperature for 2 hrs.

b. Conjugation of FAM-GAPDH-siRNA and SPACE

A 10 mM SPACE-Peptide solution was incubated with a 10 mM solution of N-(3-dimethylaminopropyl)-N′-ethylcarbodiimide hydrochloride (EDAC, Sigma) and a 9.5 mM solution of N-hydroxysulfosuccinimide sodium salt (NHS, Sigma) in equal parts in MES buffer (pH 5.5) for 15 min. The amine modified FAM-GAPDH-siRNA dissolved in MES buffer (pH 5.5) was then added to the mixture to conjugate with the peptide and allowed to mix overnight.

c. Preparation of DOTAP-Ethosomes Containing FAM-GAPDH-siRNA or FAM-GAPDH-siRNA-SPACE Conjugation

For 1 ml of ethosomes, 10 mg of DOTAP and 2 mg of cholesterol was dissolved in 2 ml ethanol and added into above obtained solution of SPACE-POPE conjugation solution. The solvent (both ethanol and water) system was removed using a rotary evaporator at room temperature. Afterwards, 1 ml of Ethanol/acetic acid buffer (45%, v/v) mixture, which contained 25 nmol of GAPDH-siRNA and 50 mg of free SPACE-Peptide, or 1 ml of Ethanol/MES buffer (45%, v/v), which contained 25 nmol of GAPDH-siRNA-SPACE conjugation and 50 mg of free SPACE, was used to hydrate the lipid film. The obtained ethosomal solution was extruded 21 times through a 100 nm polycarbonate membrane using a mini-extruder.

The detailed compositions of the DOTAP-Ethosomes are presented in Tables 3 and 4.

TABLE 3
Composition of DOTAP-Ethosomes
containing FAM-GAPDH-siRNA

| | |
|---|---|
| | Amount for 1 ml of DOTAP- |
| Composition | Ethosomes |

| POPE-NHS | 2 | mg |
|---|---|---|
| SPACE-Peptide (TFA salt) | 2 | mg |
| PBS buffer salts | 0.5 ml, 100 mM, pH 8.0 (water | |
| (left from conjugation reaction) | were removed during rotation | |
| | evaporation, salts were left) | |
| DOTAP | 10 | mg |
| Cholesterol | 2 | mg |
| Acetic acid | 400 μL (40 mM) | |
| HCl | 47 μL (1N) | |
| DI-water | 136 | μL |
| Ethanol | 447 μL, 99.9% | |
| FAM-GAPDH-siRNA | 25 | nmol |
| Free SPACE-Peptide (TFA salt) | 50 | mg |

TABLE 4
Composition of DOTAP-Ethosomes Containing
FAM-GAPDH-siRNA-SPACE conjugation

| | Amount for 1 ml of DOTAP- | |
|---|---|---|
| Composition | Ethosomes | |
| POPE-NHS | 2 | mg |
| SPACE-Peptide (TFA salt) | 2 | mg |
| PBS buffer salts | 0.5 ml, 100 mM, pH 8.0 (water | |
| (left from conjugation reaction) | were removed during rotation | |
| | evaporation, salts were left) | |
| DOTAP | 10 | mg |
| Cholesterol | 2 | mg |
| MES buffer (for dissolving siRNA) | 92 μL (25 mM, pH 5.5) | |
| SPACE (for siRNA-SPACE | 146 μL, 10 mM in MES buffer | |
| conjugation) | (pH 5.5) | |
| EDAC (for siRNA-SPACE | 146 μL, 10 mM in MES buffer | |
| conjugation) | (pH 5.5) | |
| NHS (for siRNA-SPACE conjugation) | 146 μL, 10 mM in MES buffer | |
| | (pH 5.5) | |
| FAM-GAPDH-siRNA | 25 | nmol |
| (for siRNA-SPACE conjugation) | | |
| HCl | 53 μL (1N) | |
| Ethanol | 447 μL, 99.9% | |
| Free SPACE-Peptide (TFA salt) | 50 | mg |

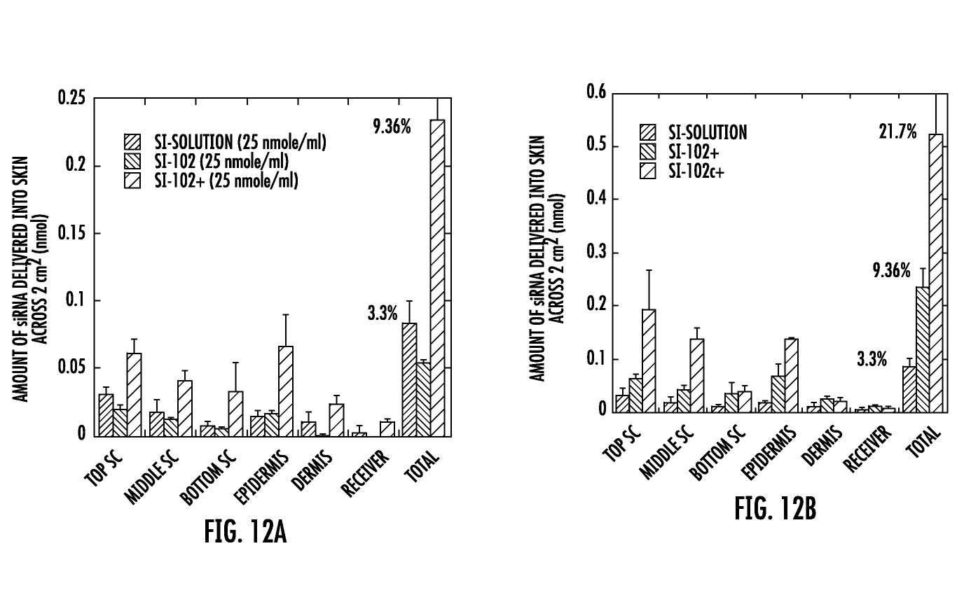

FIGS. 12A and 12B show the efficacy of these formulations in delivering siRNA into skin. Compared to an aqueous solution of siRNA, SPACE-ethosomal siRNA exhibited high penetration into skin (see FIG. 12A). Specifically, while siRNA aqueous solution exhibited only 3.3% penetration into skin, SPACE-ethosomal siRNA (SI-102+) exhibited 9.3% penetration into skin. The efficacy of penetration was even higher when SPACE-conjugated siRNA was encapsulated in SPACE-ethosomes (SI-102c+; see FIG. 12B). In this case, 21.7% of the applied dose (100 microliters of 25 nmole/ml) entered the skin through an area of 2 sq. cm in 24 hours.

### REFERENCES

All references listed in the instant disclosure, including but not limited to all patents, patent applications and publications thereof, scientific journal articles, and database entries (including but not limited to GENBANK® biosequence database entries and all annotations available therein) are incorporated herein by reference in their entireties to the extent not inconsistent herewith and to the extent that they supplement, explain, provide a background for, or teach methodology, techniques, and/or compositions employed herein.

It will be understood that various details of the presently disclosed subject matter can be changed without departing from the scope of the presently disclosed subject matter. Furthermore, the foregoing description is for the purpose of illustration only, and not for the purpose of limitation.

1. A composition comprising a peptide, an active agent, and a carrier comprising the active agent, wherein:
(a) the peptide comprises an amino acid sequence set forth in any of SEQ ID NOs: 1-18;
(b) the peptide is associated with and/or conjugated to the active agent, the carrier, or both;
(c) the carrier is selected from the group consisting of a micelle, a liposome, an ethosome, and combinations thereof; and
(d) the composition is capable of penetrating a stratum corneum (SC) layer when contacted therewith or penetrating a cell when contacted therewith,
and optionally wherein the composition further comprises one or more free peptides comprising an amino acid sequence set forth in any of SEQ ID NOs: 1-18.

2. The composition of claim 1, wherein the composition is capable of penetrating the SC layer and penetrating the cell.

3. The composition of claim 1, wherein the peptide is a cyclic peptide comprising (i) an amino acid sequence as set forth in any of SEQ ID NOs: 7-18;
and (ii) a Cys-Cys disulfide bond.

4. The composition of claim 1, wherein the composition is capable of penetrating the cellular membrane of viable non-human animal cells; viable human cells; viable epidermal or dermal cells; and/or viable immunological cells.

5. The composition of claim 1, wherein the active agent comprises a macromolecule, optionally a protein, a nucleic acid, a pharmaceutical compound, a detectable moiety, a small molecule, and/or a nanoparticle.

6. The composition of claim 1, wherein the protein comprises an antibody or a fragment thereof comprising at least one paratope.

7. The composition of claim 6, wherein the macromolecule comprises a nucleic acid.

8. The composition of claim 7, wherein the nucleic acid is DNA.

9. The composition of claim 7, wherein the nucleic acid is RNA, optionally an interfering RNA, further optionally an shRNA, an miRNA, or an siRNA.

10. The composition of claim 9, wherein the siRNA is designed to interfere with expression of a gene product selected from the group consisting of an IL-10 gene product, an IL-4 gene product, an CD86 gene product, a KRT6a gene product, a TNFR1 gene product, and a TACE gene product.

11. The composition of claim 9, wherein the siRNA is a mutation-specific siRNA.

12. The composition of claim 5, wherein the pharmaceutical compound is cyclosporin A (CsA) or hyaluronic acid (HA).

13. The composition of claim 12, wherein the pharmaceutical compound is CsA, the CsA is encapsulated by the carrier, and the peptide is conjugated to the carrier.

14. The composition of claim 13, wherein the carrier is an ethosome and the composition further comprises one or more free peptides comprising an amino acid sequence set forth in any of SEQ ID NOs: 1-18.

15. The composition of claim 5, wherein the active agent comprises a detectable agent, optionally a fluorescent label or a radioactive label.

16. A composition comprising a peptide, an active agent, and a carrier comprising the active agent, wherein:
(a) the peptide comprises an amino acid sequence set forth in any of SEQ ID NOs: 1-18;
(b) the peptide is associated with an active agent and/or a carrier comprising the active agent, wherein the association results from hydrophobic, electrostatic or van der Walls interactions;
(c) the carrier is selected from the group consisting of a micelle, a liposome, an ethosome, and combinations thereof; and
(d) the composition is capable of penetrating a stratum corneum (SC) layer when contacted therewith or penetrating a cell when contacted therewith,
and further wherein the composition optionally comprises one or more free peptides comprising an amino acid sequence set forth in any of SEQ ID NOs: 1-18.

17. The composition of claim 16, wherein the peptide is a cyclic peptide comprising (i) an amino acid sequence as set forth in any of SEQ ID NOs: 7-18, and (ii) a Cys-Cys disulfide bond.

18. A method for delivering an active agent to a subject, comprising administering to the subject a composition comprising a peptide comprising an amino acid sequence set forth in any of SEQ ID NOs: 1-18, wherein:
(i) the peptide is conjugated to an active agent or an active agent carrier comprising the active agent and/or is associated with an active agent and/or a carrier comprising the active agent, wherein the association results from hydrophobic, electrostatic or van der Walls interactions;
(ii) the carrier is selected from the group consisting of a micelle, a liposome, an ethosome, and combinations thereof; and
(iii) the composition is capable of penetrating the stratum corneum (SC) of the subject or penetrating a cell of the subject,
and optionally wherein the composition further comprises one or more free peptides comprising an amino acid sequence set forth in any of SEQ ID NOs: 1-18.

19. The method of claim 18, wherein the composition is formulated for topical administration.

20. The method of claim 18, wherein the peptide is a cyclic peptide comprising (i) an amino acid sequence as set forth in any of SEQ ID NOs: 7-18; and (ii) a Cys-Cys disulfide bond.

21-72. (canceled)


SEQUENCE LISTING (19 sequences)

SEQ ID NO: 1 - protein, 7 aa, Artificial Sequence
  Artificially synthesized peptide
TGSTQHQ

SEQ ID NO: 2 - protein, 7 aa, Artificial Sequence
  Artificially synthesized peptide
HSALTKH

SEQ ID NO: 3 - protein, 7 aa, Artificial Sequence
  Artificially synthesized peptide
KTGSHNQ

SEQ ID NO: 4 - protein, 7 aa, Artificial Sequence
  Artificially synthesized peptide
MGPSSML

SEQ ID NO: 5 - protein, 7 aa, Artificial Sequence
  Artificially synthesized peptide
TDPNQLQ

SEQ ID NO: 6 - protein, 7 aa, Artificial Sequence
  Artificially synthesized peptide
STHFIDT

SEQ ID NO: 7 - protein, 9 aa, Artificial Sequence
  Artificially synthesized peptide
CTGSTQHQC

SEQ ID NO: 8 - protein, 9 aa, Artificial Sequence
  Artificially synthesized peptide
CHSALTKHC

SEQ ID NO: 9 - protein, 9 aa, Artificial Sequence
  Artificially synthesized peptide
CKTGSHNQC

SEQ ID NO: 10 - protein, 9 aa, Artificial Sequence
  Artificially synthesized peptide
CMGPSSMLC

SEQ ID NO: 11 - protein, 9 aa, Artificial Sequence
  Artificially synthesized peptide
CTDPNQLQC

SEQ ID NO: 12 - protein, 9 aa, Artificial Sequence
  Artificially synthesized peptide
CSTHFIDTC

SEQ ID NO: 13 - protein, 11 aa, Artificial Sequence
  Artificially synthesized peptide
ACTGSTQHQCG

SEQ ID NO: 14 - protein, 11 aa, Artificial Sequence
  Artificially synthesized peptide
ACHSALTKHCG

SEQ ID NO: 15 - protein, 11 aa, Artificial Sequence
  Artificially synthesized peptide
ACKTGSHNQCG

SEQ ID NO: 16 - protein, 11 aa, Artificial Sequence
  Artificially synthesized peptide
ACMGPSSMLCG

SEQ ID NO: 17 - protein, 11 aa, Artificial Sequence
  Artificially synthesized peptide
ACTDPNQLQCG

SEQ ID NO: 18 - protein, 11 aa, Artificial Sequence
  Artificially synthesized peptide
ACSTHFIDTCG

SEQ ID NO: 19 - RNA, 21 nt, Artificial Sequence
  Artifically synthesized ribooligonucleotide
gacguaaacggccacaaguuc